(19) United States
(12) Patent Application Publication
(10) Pub. No.: US 2004/0142891 A1
(43) Pub. Date: Jul. 22, 2004

(54) Genes involved in immune related responses observed with asthma

(75) Inventors: Pieter Cornelis Groot, Den Haag (NL); Bram Jeroen van Bergenhenegouwen, Utrecht (NL); Antonius Josephus Maria van Oosterhout, Utrecht (NL)

Correspondence Address: TRASK BRITT, P.O. BOX 2550, SALT LAKE CITY, UT, 84110, US

(73) Assignee: Universiteit Utrecht, Utrecht (NL)

(21) Appl. No.: 10/677,856

(22) Filed: Oct. 2, 2003

Related U.S. Application Data
(63) Continuation-in-part of application No. 10/369,214, filed on Feb. 15, 2003.
Application No. 10/369,214 is a continuation of application No. PCT/NL01/00610, filed on Aug. 16, 2001.

(51) Int. Cl. A61K 48/00; C12Q 1/68

(52) U.S. Cl. 514/44; 435/6

(57) ABSTRACT

Asthma is one of the most common chronic diseases (155 million people worldwide) and is rapidly increasing (20-50% per decade), particularly in children (currently 10% in The Netherlands). Asthma impairs the quality of life and is a major cause of absence from school and work. Asthma, if not treated properly, can be life threatening. The invention provides a nucleic acid library comprising genes or functional fragments thereof wherein the genes are essentially capable of initiation and/or progression and/or suppression and/or repression of an immune response.

### CROSS-REFERENCE TO RELATED APPLICATIONS

This application is a continuation-in-part of pending appln. U.S. Ser. No. 10/369,214, filed Feb. 15, 2003, which is a continuation of appln. No. PCT/NL01/00610, filed Aug. 16, 2001, designating the United States of America (published in English as PCT International Publication No. WO 02/14366, Feb. 21, 2002), the contents of all of which are incorporated by this reference.

### TECHNICAL FIELD

The invention relates to the field of immunology, gene therapy and medicine. Asthma is one of the most common chronic diseases (155 million people worldwide) and is rapidly increasing (20-50% per decade), particularly in children (currently 10% in The Netherlands). Asthma impairs the quality of life and is a major cause of absence from school and work. Asthma, if not treated properly, can be life threatening.

### BACKGROUND

Allergic asthma can be characterized by reversible airway obstruction, elevated levels of IgE, chronic airway inflammation and airway hyperresponsiveness to bronchoconstrictive stimuli, airway tissue remodeling and mucus hypersecretion. The allergic inflammatory infiltrate in the airway tissue predominantly consists of eosinophils and CD4⁺ T-lymphocytes. It is now widely accepted that type 2 T-helper (Th2) lymphocytes which produce a limited set of cytokines including interleukin-3 (1L3), 1L4, 1L5, IL9, ILIO and IL13 play an important role in the initiation and progression of allergic asthma [Corrigan and Kay (1992). Immunology Today. 13, 501-507; Roinagnani, S. (2000) J Allergy Clin Immunol 105, 399-408]. Chronic asthma appears to be driven and maintained by persistence of a subset of chronically activated memory T-cells (lymphocytes). Besides T-lymphocytes many other inflammatory cell-types are involved in the pathophysiology of allergic asthma such as eosinophils, mast cells, B-lymphocytes, dendritic cells, macrophages and monocytes as well as resident airway cells such as epithelial cells and smooth muscle cells. Moreover, sensory neurons of which the cell bodies are located in the dorsal root ganglia play an important role in airway inflammation, hyperresponsiveness and cough.

Currently used pharmacological therapies in allergic asthma only provide temporal symptomatic relief. A more findamental treatment aimed at antigen-specific T-lymphocytes and antigen-presenting cells is desirable since these cell-types play a crucial role in the initiation and progression of allergic asthma. Furthermore, T-lymphocytes may be the only cells that have the potential to induce long-term relieve of symptoms. Current therapy for moderate to severe asthma essentially involves multiple classes of molecules: anti-inflammatory glucocorticoids, bronchodilator drugs, and mast-cell inhibitors. The current preferred method is to treat the chronic phase of asthmatic symptoms, as manifested by airway hyperresponsiveness and eosinophilic inflammation, with glucocorticoids to reduce the inflammatory component and hyperresponsiveness (Barnes, 1990; Schleimer, 1990). These drugs are not very selective, targeting non-inflammatory cells as well as inflammatory cells and often have moderate to serious side effects after chronic treatment, especially in children. Furthermore, a subgroup (10%) of asthma patients become relatively resistant to glucocorticoid therapy and increasingly become dependent upon non-glucocorticoid treatment. In addition, there is a strong need for so-called “add-on” therapies to limit the use of high doses of glucocorticoids and the associated side effects. Hence, there is a strong need for a safer, more selective and more efficacious therapeutic which displays a long-term clinical benefit to asthma patients.

The invention provides a nucleic acid library comprising genes or functional fragments, derivatives or analogues thereof essentially capable of modulating an immune response observed with airway hyper-responsiveness and/or bronchoalveolar manifestations of asthma. Modulation herein can refer to up-regulation or down-regulation of an immune response, for example by activation and/or suppression of gene(s) which are essentially capable of initiation and/or progression and/or suppression and/or repression of an immune response and/or symptoms of said immune response. Modulation herein can also refer, for example to positive (i.e up-regulation) or negative (i.e down-regulation) regulation of gene transcription, and to the modulation of the gene and gene product. Methods for modulating the expression of genes and gene products are known. The definition functional fragment thereof means that a particular subject sequence may vary from the reference sequence by one or more substitutions, deletions, or additions, the net effect of which does not result in an adverse functional dissimilarity between the reference and the subject sequence. An analogue is a compound having functional equivalence or being related to a molecule in question. The invention provides a nucleic acid library comprising nucleic acid or functional fragments, derivatives or analogues thereof comprising at least one gene as listed in table 1, 2 or 3, genes which play an important role in all immune system related disorders such as all allergic diseases (asthma, rhinitis, atopic dermatitis, urticaria) and auto-immune diseases (i.e multiple sclerosis). The invention provides a nucleic acid library comprising such genes or fragments thereof said genes essentially capable of modulating an immune response observed with airway hyperresponsiveness and/or bronchoalveolar manifestations of asthma wherein said immune response is up-regulated and/or down-regulated. An immune response herein refers to the physiological response(s) stemming from activation of the immune system by antigens, including immunity to pathogenic organisms and auto-immunity to self-antigens, allergies, inflammatory response and graft rejection. An immune response herein further applies to all immune system related disorders. Usually the antigenic invader comprises a protein or protein attached moiety. The invention further provides a library comprising genes or functional fragments derivatives or analogue thereof said genes essentially capable of initiation and/or progression (i.e. up-regulation) and/or suppression and/or repression (down-regulation) of an immune response wherein said immune response are airway hyperresponsiveness and/or broncheoalveolar manifestations of asthma. The invention provides a nucleic acid or functional fragments thereof selected from those listed in table 1, 2 or 3, capable of initiation and/or progression and/or suppression and/or repression of an immune response wherein said immune response is asthma. Methods of detecting nucleic acids capable of initiation and/or progression and/or suppression and/or repression of an immune response are known. In one embodiment such a nucleic acid is derived from a DC-SIGN gene is described herein. DC-SIGN (signature sequence OtS1-B7) in the primary cultures of bone-marrow derived dendritic cells demonstrates an important role of this gene and the encoding protein in the cellular function of dendritic cells. Bone-marrow derived dendritic cells or cell-lines representing dendritic cells such as XS52 cell-line or other primary cell cultures of this cell-type can be used to determine the gene/protein function and screening of a compound (agonist or antagonist) that modulates at least one of the functions of the gene/protein. Dendritic cells are so-called professional antigen-presenting cells (APC) and thus play a crucial role in the initiation and progression of immune- and inflammatory responses mediated by T-lymphocytes. Blockade of mDC-SIGN/OtS1-B7 is beneficial in the treatment of T-lymphocyte mediated diseases such as allergy, asthma, COPD, auto-immune diseases, inflammatory bowel diseases, allograft rejection and infectious diseases. One of the herein disclosed examples shows that blocking of OtS1-B7 in an asthmatic mouse model results in decreased serum levels of IgE and a decrease in airway hyperreactivity. Blocking of OtS1-B7 is for example accomplished by providing an (monoclonal) antibody such as ERTR-9. Allergic diseases are typically accompanied by enhanced levels of (serum) IgE and although IgE can have beneficial effects, for example against parasitic infections, high levels of IgE are considered to be very detrimental for the health. Hence the invention provides a method for at least in part decreasing at least one allergy symptom in a mammal suffering from allergy, comprising administering to said mammal a substance capable of blocking OtS1-B7 or an equivalent of OtS1-B7. Increased (serum) IgE levels also play a role in asthma and hence the invention provides a method for at least in part decreasing at least one asthma symptom in a mammal having said at least one asthma symptom, comprising administering to said mammal a substance capable of blocking OtS1-B7 or an equivalent of OtS1-B7. The term “OtS1-B7 or an equivalent of OtS1-B7” is herein defined as protein(fragment) encoded by a mouse gene with the signature sequence OtS1-B7 or an equivalent thereof in another mammal, for example a human homologue of the mouse gene with the signature sequence OtS1-B7. Preferably, the blocking substance is a proteinaceous substance and even more preferably said proteinaceous substance is a(n) (monoclonal) antibody or a functional equivalent and/or a functional fragment thereof. Polyclonal antibodies and monoclonal antibodies may be produced by methods known to the person skilled in the art. Moreover, a(n) (monoclonal) antibody may further be adapted to the mammal that is in need for treatment. For example, said antibodies may be humanised by known methods. A functional fragment of an antibody is for example the Fab-fragment. A functional equivalent is for example a slightly modified antibody by way of deletion or insertions or for example by combining the Fab part of an antibody with another Fc-part. An example of an antibody that is used in the method according to the invention is the monoclonal antibody ERTR9 or a functional equivalent an/or functional fragment thereof An example of a functional equivalent is a (monoclonal) antibody raised against the protein(fragment) encoded by the human homologue of the mouse gene with signature sequence OtS1-B7. Yet other examples of a substance capable of blocking OtS1-B7 or an equivalent of OtS1-B7 are small peptides or small (organic or synthetic) molecules. In yet another embodiment the gene with the signal sequence OtS1-B7 or an equivalent of OtS1-B7 is blocked by anti-sense technology.

In yet another preferred embodiment, the invention further provides a composition or a pharmaceutical composition comprising a substance capable of blocking OtS1-B7 or an equivalent of OtS1-B7 and a (pharmaceutical acceptable) carrier and/or diluent. More preferably, said substance is a proteinaceous substance, a (small) peptide or a synthetic molecule. Even more preferably, said proteinaceous substance is an antibody or a functional equivalent and/or a functional fragment thereof and yet even more preferably, said antibody is ERTR9 or a functional equivalent and/or a functional fragment thereof.

Such a (pharmaceutical) composition is very useful in the treatment of a mammal suffering from an allergy and/or an asthma symptom. It is clear that said substance may be delivered by a variety of possible routes, for example via intraperitoneal or intravenous injection, orally or by inhalation. Moreover, the substance may be part of pharmaceutical composition that further comprises a pharmaceutical suitable and/or acceptable carrier or diluent. A person skiled in the art is aware how to select the proper carrier and/or diluent. The method according to the invention may be used to treat different kinds of asthma symptoms, for example but not limited to the treatment of an elevated level of IgE or treatment of airway hyperreactivity. The method according to the invention is furthermore used to decrease the (serum) IgE level in a mammal that suffers from an allergy.

Furthermore, the method according to the invention is typically applied to a human. Preferably, the invention provides a method for at least in part decreasing asthma symptoms in a mammal suffering from said symptoms, comprising providing said mammal with a substance capable of blocking OtS1-B7 or an equivalent of OtS1-B7, wherein said symptoms are (completely) decreased. Even more preferably, the (partial) decrease in asthma symptoms results in an improved sense of overall well-being. Non-limiting examples of asthma symptoms that can be treated according to a method of the invention include reversible airway obstruction, elevated levels of IgE, chronic airway inflammation and airway hyperresponsiveness to bronchoconstrictive stimuli, airway tissue remodeling and mucus hypersecretion. The method according to the invention is furthermore used to decrease the (serum) IgE levels in patients suffering from allergy. Non-limiting examples of allergies are allergy to dust, allergy to grasses or allergy to certain food ingredients.

Furthermore, the invention also provides use of an OtS1-B7 blocking substance or the use of an OtS1-B7 blocking substance for the preparation of a medicament for the treatment of allergy and/or asthma.

In another embodiment, such a gene is derived from a calcium-activated chloride channel gene as also described below. Calcium-activated chloride channels (CLCA1-4) can be blocked by mono- and polyclonal antibodies or fragments thereof directed against the ion channel (protein or peptide fragments); known non-specific chloride channel antagonists such as 4,4′-diisothiocyanatostilbene-2,2′-disulphonic acid (DIDS), 4-acetamido-4′-isothiocyanostilbene-2,2′-disulfonic acid (SITS), 5-nitro-2-(S-phenylpropylamino)benzoic acid (NPPD), niflumic acid, and the anti-allergic drug cromolyn

Changes in gene expression underlie most, if not all, pathophysiological processes. A variety of methods for detecting changes in gene expression in a healthy versus a diseased animal to detect nucleic acid for the formation of a library the subject of the invention are known. These procedures include, but are not limited to DNA-DNA or DNA-RNA hybridisation. The form of such quantitative methods may include, Southern or Northern analysis, dot/slot blot or other membrane based technologies; PCR technologies such as DNA Chip, Taqman®, NASBA, SDA, TMA, in-situ-hybridisation, protein bioassay or immunoassay techniques ELISA, IFA, proteomic and metabolomic technologies. These technologies are often found at the basis of commercially available diagnostic kits often used for screening purposes.

The invention provides a nucleic acid library comprising genes or fragments thereof said genes essentially capable of modulating an immune response observed with airway hyperresponsiveness and/or bronchoalveolar manifestations of asthma wherein said genes comprises a nucleic acid essentially equivalent to a signature sequence as shown in table 1, 2 or 3. A signature sequence herein refers to a marker sequence and/or sequence or any other mode of identification of a sequence (i.e name). Nucleic acid sequence as used herein refers to an oligonucleotide, nucleotide or polynucleotide, and fragments or portions thereof, and to DNA or RNA of genomic or synthetic origin which may be single- or double-stranded, and represents the sense or antisense strand. The definition ‘antisense’ RNA is an RNA sequence which is complementary to a sequence of bases in the corresponding mRNA: complementary in the sense that each base (or majority of bases) in the antisense strand (read in the 5′ to 3′ sense) is capable of pairing with the corresponding base (G with C, A with U), in the mRNA sequence read in the 5′ to 3′ sense. The definition ‘sense’ RNA is an RNA sequence which is substantially homologous to at least part of the corresponding mRNA sequence. Preferably the nucleic acid is an ‘immune response gene’. An immune response gene is any gene that determines the ability of lymphocytes to mount an immune response to specific antigens. The definition ‘essentially equivalent’ means that the subject signature sequence can vary from the reference sequence by one or more substitutions, deletions, or additions, the net effect of which will not result in a functional dissimilarity between the two sequences. It may be advantageous to produce nucleotide sequences, the subject of the invention or derivatives thereof possessing a substantially different codon usage. It is known by those skilled in the art that as a result of degeneracy of the genetic code, a multitude of gene sequences, some bearing minimal homology to the nucleotide sequences of any known and any naturally occurring genes may be produced. The invention includes each and every possible variation of the nucleotide sequences that could be made by selecting combinations based on possible codon choices.

The invention provides a library wherein said genes encode a regulatory molecule and/or co-stimulatory molecule and/or adhesion molecule and/or receptor molecule involved in modulating an immune response. The definition ‘regulatory molecule’ is an entity which assists the cell in ‘sensing’ it's environment. For example ‘a regulatory molecule’ can effect a immune response by modulating either positively or negatively gene transcription. The definition ‘stimulatory molecule’ is an entity which can activate an immune response. The definition ‘adhesion molecules’ is any pair of complementary molecules that bind specifically to one another to effect a positive or negative immune response. The molecule can be any entity which can bind to for example nucleic acid, proteinaceous substance or receptor etc., to effect a positive or negative immune response. The definition ‘receptor’ is an entity to which a ligand binds which triggers an immune response. The definition ‘receptor molecule’ could be for example a ligand (i.e any macromolecule) which binds to a receptor to effect an immune response. A ligand is a molecule that binds to a complementary site on a given structure. For example oxygen is a ligand for haemoglobin and a substrate of an enzyme molecule is a specific ligand of that molecule. The invention further provides a method for modulating an immune response of an individual comprising modulating a gene comprising a nucleic acid at least functionally equivalent to a nucleic acid identifiable by a signature sequence as shown in table 1, 2 or 3.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example Gob-5 (signature sequence R1-SO-R1-C11). Gob 5 is a cell-membrane protein belonging to the family of calcium-activated chloride channels and discovered in intestinal goblet cells in mice. Human CaCC1 and the identical CLCA1 are most likely the human homologues of murine gob-5. Gob-5 can have another function as a cell adhesion molecule. Northern blot analysis revealed that gob-5 is abundantly expressed in the stomach, small intestine, uterus and slightly expressed in the trachea of mice. In-situ hybridization demonstrated that gob-5 expression is located in the mucus-secreting cells of these three tissues. In humans, CaCC1/CLCA1 are also primarily expressed in the digestive tract Gob-5 is expressed in lymph-nodes, lung tissue, bronchoalveolar lavage cells and bone-marrow from mice and is up-regulated in these tissues in the mouse asthma model. Mucus secreting goblet cells have never been described in lymph nodes or bone-marrow. In addition, Gob 5 is expressed in murine bone-marrow derived mast cells and murine mast-cell lines. Gob-5 plays a role in secretory processes based on its fction as a chloride channel. Chloride channels have been shown to be involved in mast cell activation and degranulation since inhibition of these channels by non-selective broad spectrum chloride channel inhibitors inhibit IgE -mediated rat mast-cell degranulation in-vitro. Additionally a strong up-regulation of gob-5 in the dorsal root ganglia (DRG) in the mouse asthma model was observed. The expression of other members of the calcium-activated chloride channel gene family by PCR (table 2) was investigated. Murine homologue of human CaCC3 (EST AA726662) was identified and their expression was shown to be strongly upregulated in DRG of the mouse asthma model.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example LR8 (R1-OS-B1-D3). LR8 belongs to the family of the tetraspanin (4TM) superfamily. LR8 mRNA was not detectable by PCR in human smooth muscle cells, endothelial cells or epithelial cells. Murine LR8 mRNA expression in lymph nodes from mice was confirmed along with a down-regulation in a mouse asthma model. Bio-informatics analysis of the LR8 protein confirmed the presumed 4TM structure of the protein and revealed a striking homology with the beta chain of the high affinity IgE receptor (FceRI). The tetraspanin superfamily has grown to nearly 20 known genes since its discovery in 1990. All encode ceu-surface proteins that span the membrane four times, forming two extracellular loops. Many of these proteins have a flair for promiscuous associations with other molecules, including lineage-specific proteins, integrins, and other tetraspanins. In terms of function, they are involved in diverse processes such as cell activation and proliferation, adhesion and motility, differentiation, and cancer. These functions relate to their ability to act as “molecular facilitators,” grouping specific cell-surface proteins and thus increasing the formation and stability of functional signaling complexes. LR8 is similar to CLAST1, a murine gene that is activated upon ligation of CD40 (Genbank: BAA88596). CD40 is predominantly expressed on so-called “antigen-presenting cells” and ligation of CD40 induces the expression of several molecules involved in the activation and regulation of T-lymphocytes (CD80; CD86; IL12). CD40 is an important maturation signal for dendritic cells. Immature dendritic cells take up antigen in peripheral tissues and migrate to secondary lymphoid tissues (draining lymph node) where they maturate and present antigen to lymphocytes. Several proteins are induced or down-regulated upon dendritic cell maturation. Many of the differentially activated genes appear to be involved in the modulation (regulation/activation) of T-lymphocytes (table 1, 2 or 3).

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example β-Amyloid-precursor like protein 2 (APLP2) (SvO2-1-B7). APLP2 is a highly conserved protein and is located on mouse chromosome 9. Moreover, in an experimental asthma model, airway hyper-responsiveness has been linked to a locus on chromosome 9, syntenic with human 11q24. APLP2 is a member of the Alzheimer precursor protein family including the Alzheimer peptide precursor (APP). These proteins all share three domains of similarity, interdispersed with completely divergent regions. APLP2 is a type-I integral membrane protein that contains a single membrane spanning domain with a large extracellular N-terminal domain and a short C-terminal cytoplasmic domain. APPL2 is ubiquitously expressed. Alternative splicing of APPL2 pre-mRNA generates at least four transcripts. Several functional domains have been identified in APLP2, including a DNA binding motif, an N-terminal cysteine rich domain exhibiting zinc, copper, and heparin binding activity, followed by a very acidic region and, depending on the isoform, the Kunitz protease inhibitor (KPI) domain. Interestingly, the KPI domain inhibits serine proteases like trypsin, plasmin, tryptase and chymase of which the latter two are released by activated mast cells. Tryptase has been implicated in the development of airway hyperresponsiveness. Mast cell mediator serotonin stimulates the release of APLP2 ectodomain (containing the KPI domain). Other functions that have been described for APLP2 are (i) an interaction with MHC class I, (ii) a role as adhesion molecule through interactions with extracellular matrix components, (iii) a role in epithelial wound healing and (iv) a potential role in the inhibition of platelet activation by the N-terminal cysteine-rich domain.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. For example the invention provides a method for the treatment of an immune response more particularly asthma and COPD comprising providing APLP2 or its KPI domain or by induction of APLP2 expression. APLP2 through the inhibition of the detrimental effects of mast cell proteases, by repair of epithelial damage and by inhibition of platelet activation is capable of treating an immune related response. Furthermore, many allergens have been shown to have protease activities that appear to be crucial for allergic sensitization. By its KPI domain, APLP2 can inhibit the proteolytic activities of allergens and thereby prevent the initiation and progression of allergic responses. Another effect of the KPI domain of APLP2 is inhibition of the activation of protease-activated receptors (PARs) by serine proteases. PAR2 is involved in bronchorelaxation and protection against bronchoconstriction by stimulating the generation of prostaglandin E2 by airway epithelial cells. However, it was demonstrated that trypsin and a PAR2 ligand induced bronchoconstriction in guinea pigs in vivo, despite the induction of relaxation by these mediators in isolated trachea and bronchi. The bronchoconstriction appeared to be mediated by a neural mechanism since the bronchoconstriction was inhibited by the combination of NK1 and NK2 receptor antagonists. These data suggest that the PAR2 ligand activates sensory nerves. In agreement herewith, trypsin and mast cell tryptase induced a wide-spread neurogenic inflammation initiated by activation of neuronal PAR2 receptors. Inhibition of tryptase and other serine proteases by APLP2 or its KPI domain can antagonize neurogenic inflammation and bronchoconstriction. Moreover, other PARE appear to be involved in inflammation. Activation of these receptors (PAR2) by serine proteases is sensitive to inhibition by APLP2 or its KPI domain. Analogous to intra-membrane cleavage of APP and Notch by aspartyl proteases (γ-secretase, presenilins). APLP2 can be cleaved by these aspartyl proteases since it is homologous to APP in the region (IATVIVI) where y-secretase cleaves APP. This cleavage will lead to the generation of the extracellular part of APLP2 and an intracellular part of 57 amino acids, which may directly or indirectly modify the transcription of target genes. The APLP2 C57 peptide contains the “NPTY” sequence, which is present in many growth factor receptors and appears to be involved in cellular signaling. Interestingly, T-lymphocytes have been shown to express presenilin-1 and 2 at the cell-surface. Cleavage of APLP2 is involved in T-lymphocyte activation. Another, at present unidentified protease may cleave APLP2 in its transmembrane region and generate the release of an intracellular peptide containing the “NPTY” sequence.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. For example phosphotyrosine binding (PTB) domains have been identified in a large number of proteins. PTB domains play an important role in signal transduction by growth factor receptors. Several PTB proteins have been shown to bind to amyloid proteins through the “NPTY” motif like Fe65, Fe65-like, X11 and X11-like proteins, Shc and IRS-1. The interactions of APLP2 with Shc and IRS-1 is dependent on tyrosine phosphorylation whereas the interactions with Fe65 and X11 are not. The Fe65 adaptor protein interacts with the transcription factor CP2/LSF/LBP1. The “NPTY” motif, has been shown to be involved in binding to Shc, a Src homology 2 (SH2)-containing proto oncogene product implicated in activating Ras via association with Grb2 protein. Activation of the Ras pathway involves the MAPK signal transduction pathway which has been shown to be involved in the induction of many inflammatory genes. The Shc/Grb2/Sos complex is also involved in the activation of the Ras pathway in T-lymphocytes. It is unknown whether APLP2 or other proteins of this family with an “NPTY” domain are involved in T-cell activation and differentiation. Caspases can also cleave APP at the caspase consensus site “VEVD”, leading to the generation of a C-terminal 31 amino acid peptide which contains the internalization sequence “NPTY”. Since APLP2 contains both the caspase consensus site “VEVD” as well as the internalization sequence “NPTY”, it is clear that APLP2 can also be cleaved by caspases leading to the generation of a C-terminal 31 amino acid peptide which is homologous to the peptide generated by APP cleavage. The APP C31 peptide has been demonstrated to initiate cell death. Apoptosis or cell-death is an important mechanism to limit immune and inflammatory reactions. On the other hand, cell-death may be unwanted i.e. death of airway epithelial cells may increase airway responsiveness. The invention provides a method for the treatment and/or prevention of an immune related response more particularly allergic asthma and related inflammatory diseases and COPD comprising modulating APLP2 or its KPI domain and/or by induction of APLP2 expression. Treatment by providing APLP2 or its KPI domain or induction of APLP2 expression is effective in the treatment of (1) the neurogenic component of inflammatory responses, (2) hyperalgesia during inflammatory responses, (3) cough due to airway inflammation and (4) bronchoconstriction induced by activation of sensory nerves. Cleavage of APLP2 by presenilins (γ-secretase) or other proteases or by caspase is involved in activation-induced cell-death in T-lymphocytes and is involved in the induction of peripheral tolerance.

The invention provides a substance such as a proteinaceous substance capable of modulating a gene comprising a nueleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma for example the invention provides a method for the treatment of immune responses comprising stimulating the cleavage of the intracellular domain of APLP2 by allosteric activation of proteases or by binding of APLP2 to its ligand together with an antigen-specific stimulation which will induce peripheral tolerance to the antigen. This treatment is effective for allergic asthma and other diseases mediated by T-lymphocytes such as auto-immunity and graft-rejection.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example mouse GDP-dissociation inhibitor (Ly-GDI: signature sequence Sv-O2-1-D8). Ly-GDI was originally identified in lymphocytes and likewise called “lymphoid-speciftc GDI” (Ly-GDI). Independently, Ly-GDI gene was cloned from human and from mouse and it GDP-dissociation inhibitor D4 was designated. Mouse and human D4-GDI (Ly-GDI) share 89% amino acid sequence identity. Murine Ly-GDI is located on chromosome 6, the human homologue (Ly-GDI or D4-GDI) is located on chromosome 12p12.3. Northern blot analysis demonstrated that Ly-GDI was expressed abundantly in lung, and at lower levels in several other tissues. Another study using Northern blot analysis revealed that Ly-GDI is expressed as a 1.4-kb transcript only in hematopoietic tissues. Antibodies against Ly-GDI recognized a 27-kD protein on Western blots of B- and T-cell line lysates. It is now generally accepted that Ly-GDI is preferentially expressed in hematopotietic cells and can function as a GDP-dissociation inhibitor of Rho GTP binding proteins (Rac and Cdc42) but with less potency than the ubiquitously expressed RhoGDI. There are three subfamilies of small GTP-binding proteins, Ras, Rho and Rab. The present thinking is that Ras proteins are principally involved in signal transduction and cell proliferation, Rho proteins (Rac1, Rac2, TC10 and Cdc42) regulate cytoskeletal organization and Rab proteins are involved in the control of intracellular membrane traffic. The GTP-binding proteins are active only in the GTP-bound state. At least 2 classes of proteins tightly regulate cycling between the GTP-bound (active) and GDP-bound (inactive) states: GTPase-activating proteins (GAPs) and GDP/GTP exchange factors (GEF). GAPs inactivate small GTP-binding proteins by stimulating their low intrinsic GTPase activity to cause hydrolisis of GTP to GDP. GEFs are of two types including GDP dissociation stimulators (GDS, alternatively called guanine nucleotide releasing factors (GRF) and GDP-dissociation inhibitors (GDIs). The GDIs decrease the rate of GDP dissociation from Ras-like GTPases. It was found that Ly-GDI bound RhoA, and in-vitro inhibited GDP dissociation from RhoA. Stimulation of T lymphocytes with phorbol ester led to phosphorylation (activation) of Ly-GDI. It has been suggested that Ly-GDI may be involved in the regulation of hematopoietic-specific Rho-family GTPases because it is less potent than the ubiquitously expressed Rho-GDI. In T-lymphocytes, Rac and Cdc42 are important Rho-family GTPases involved in T-cell activation. Both Rac and Cdc42 are activated by Vav that has GDS activity (see FIG. 1). Rac and Cdc42 are involved in downstream signaling to the nucleus via the JNK pathway leading to the transcription factors API (fos/jun) and NFAT (nuclear factor of activated T-cells). These transcription factors are involved in transcription of cytokines such as IL1, IL4, GM-CSF etc. Recently, it was demonstrated that Ly-GDI also interacts with the proto-oncogene Vav. Vav functions as a specific GDS for Rho, Rac and Cdc42 and is regulated by tyrosine phosphorylation in hematopoietic cells. Vav integrates signals from lymphocyte antigen receptors and co-stimulatory molecules to control development, differentiation and cell cycle. Interestingly, Vav knock-out mice have a defective IgE antibody production that can be attributed to compromised T cell help due to impaired IL-4 transcription. Ly-GDI knock-out mice have been generated and did not show striking abnormalities of lymphoid development or thymocyte selection. The mice also exhibited normal immune responses including lymphocyte proliferation, IL-2 production, cytotoxic T lymphocyte activity, antibody production, antigen processing and presentation, immune cell aggregation and migration, and protection against an intracellular protozoan. However, Ly-GDI-deficient mice exhibited deregulated T and B cell interactions after in vitro cultivation of mixed lymphocyte populations in concanavalin A (Con A) leading to overexpansion of B lymphocytes. Further studies revealed that Ly-GDI deficiency decreased IL-2 withdrawal-induced apoptosis of lymph node cells while dexamethasone- and T cell receptor-induced apoptosis remained intact. These data implicate the regulation of the Rho GTPase by Ly-GDI in lymphocyte survival and responsiveness, but suggest that these functions may be partially complemented by other Rho regulatory proteins when the Ly-GDI protein is deficient Increased expression of GDP-dissociation inhibitor in the mouse asthma model in the lung-draining lymph nodes of “asthmatic” (OVA-challenged) compared to “healthy” (saline-challenged) mice was observed. A role for the GDP-dissociaton inhibitor in the generation of Th2 immiune responses is provided.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example a mouse fragment (signature sequence R1-SO-R1-A12) homologous to several mouse EST's and human (Cdc42-GAP) was identified. Human Cdc42 GTPase-activating (Cdc42-GAP) functions as a GAP for the Rho-family GTPase Cdc42 (See FIG. 1). Cdc42 can regulate the actin cytoskeleton through activation of Wiskott-Aldrich syndrome protein (WASP). Mutations in WASP lead to the Wiskott-Aldrich syndrome, a paediatric disorder characterized by actin cytoskeletal defects in heamatopoletic cells, leading clinically to thrombocytopenia, eczema and immunodeficiency. Recently, WASP-interacting protein (WIP) was shown to enhance the Vav-mediated activation of NF-AT/AP-1 gene transcription. Moreover, the interaction of WIP with WASP is necessary, but not sufficient for the ability of WIP to regulate NF-AT/AP-1 activity. Both Ly-GDI and Cdc42-GAP function in concert as inactivators of Cdc42. The invention provides a method for the treatment of immune responses more in particular allergic asthma and related allergic and Th2 -mediated inflammatory diseases comprising providing blockade of Ly-GDI and/or Cdc42-GAP by selective antagonist(s) which inhibit T-helper lymphocyte type-2 (Th2) responses. The invention provides a method for the treatment of immune responses more in particular Th1-lymphocyte mediated diseases like auto-immune diseases comprising modulating Ly-GDI and/or Cdc42-GAP, more preferably inducing the expression of these proteins. Induction of the expression of these proteins induces T-helper lymphocyte type-2 responses and is therefore effective in the treatment of Th1-lymphocyte mediated diseases like auto-immune diseases.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example TIS11d/tristetraprolin homologue (signature sequence OtS2-A7). The human TIS11d protein is part of the TIS11 family of proteins also called tristetraprolin protein. These are basic proline-rich proteins and contain an unusual CCCH type of zinc finger structure. Tumor necrosis factor-α is a major mediator of both acute and chronic inflammatory responses in many diseases. In addition to its well-known role in acute septic shock, it has been implicated in the pathogenesis of chronic processes such as autoimmunity, graft-versus-host disease, rheumatoid arthritis, Crohn's disease, and the cachexia accompanying cancer and AIDS. TIS11 interferes with TNF-α production by destabilizing its mRNA. This pathway represents a potential target for anti-TNF-α therapies. TIS11 deficiency also results in increased cellular production of granulocyte-macrophage colony-stimulating factor and increased stability of its mRNA, apparently secondary to decreased deadenylation. TIS11 is a physiologic regulator of GM-CSF mRNA deadenylation and stability. The invention provides a method for the treatment of an immune related response, comprising modulating expression, more preferably increased expression of TIS11d protein which inhibits the development of allergic asthma and related allergic and inflammatory diseases.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example many of the differentially activated genes as listed in table 1, 2 or 3 are involved in the regulation/activation of T-lymphocytes (T-lymphocyte activation molecules). Those up-regulated genes/proteins included terminal deoxynucleotidyl transferase (signature sequence: R1-SO-R1-E7), CsA-19 (signature sequence: ST-O1-B3), Pendulin (signature sequence: R1-SO-R1-E11), RA70 (signature sequence: STO1-D3), Ly-GDI (signature sequence SVO2-1-D8), Plastin-2 EST (signature sequence: SV02-1-C4), RNA Polymerase-II subunit EST (signature sequence: SV02.1-G3), Clathrin EST (signature sequence: SV02-1-D4), Cdc42-GAP (signature sequence: R1-SO-R1-A12). Those down-regulated genes/proteins were Stat-1 (signature sequence: R1-OS-B1-G3) IL2-R-gamma (signature sequence: OTS2-D9) IFN-γ-R (signature sequence: OTS2-A10).

The invention provides a method for modulating an immune response of an individual comprising modulating a gene comprising a nucleic acid at least functionally equivalent to a nucleic acid identifiable by a signature sequence as shown in table 1, 2 or 3, wherein said gene modulates a signal transduction cascade pertaining to an immune response. Method for modulating the expression of a nucleic acid are well known. In a preferred embodiment are nucleic acids as shown in table 1, 2 or 3 and functional equivalents whose products are capable of modulating genes of pathways central to immune response. Modulating herein can also mean activation or suppression. More preferable is that the nucleic acid is involved in signal transduction cascades leading to suppression or activation of an immune responses. More preferable is that the nucleic acid encodes a proteinous substance (e.g a transcription factor) which may be involved in the activation or suppression of the Ras pathway in T-lymphocytes. Activation of the RAS pathway involves the MAP kinase (MAPK) signal transduction pathway which is involved in the induction of many immune related genes.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example LR8 LR8 is part of a multi-chain Fc receptor and is involved in the signal transduction by this Fc receptor upon ligand (immunoglobulin) binding. The invention provides a method for the treatment of an immune response comprising providing blockade of LR8. Blockade of LR8 prevents the activation of inflammatory cells through this Fc receptor. The invention provides a method for the treatment and/or prevention of an immune related response comprising modulating inhibition of aspartyl proteases such as presenilins (γ-secretase) involved in the cleavage of the intracellular 57 amino-acid part of APLP2 and blockade of the “NPTY” motif, which prevents activation of downstream signal transduction pathways including the Ras and MAPK pathway and associated changes in gene expression.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example Heat-shock protein 84 (Hsp84)(signature sequence: OTS2-C6). Hsp84 is a member of the Hsp90 family of heat-shock proteins. Hsp90 proteins are ubiquitous molecular chaperones with key roles in the folding, activation and assembly of a range of client proteins typically involved in signal transduction, cell cycle control or transcriptional regulation. Hsp90 has been shown to possess an inherent ATPase activity that is essential for the activation of authentic client proteins. Recently, Hsp90 and hsc70 (signature sequence: OTS2-H2) are both necessary and sufficient to activate hormone binding by the glucocorticoid receptor. A deficiency of Hsp90 or Hsp70 proteins may thus decrease the sensitivity of cells to the effects of glucocorticoids. In asthma, a gradual decrease in glucocorticoid sensitivity occurs. This decrease in glucocorticoid sensitivity can be mimicked by several cytokines e.g. IL-4. The invention provides a method for the treatment and/or prevention of an immune related response comprising modulating expression, more preferably increased expression of Hsp90 and/or Hsp70 proteins. This increases the sensitivity to the anti-inflammatory effects of glucocorticoids and is valuable in the treatment of asthma and other chronic inflammatory diseases.

Transcription factors are directed to the nucleus by their nuclear localization sequence (NLS) in a multistep process. The first step is to dock the NLS-containing protein to the nuclear pore and this is carried out by pendulin and Srp1. Pendulin (signature sequence R1-SO-R1-E11) contains an armadillo repeat region that is involved in NLS binding. Pendulin has been shown to be involved in the nuclear localization of lymphoid enhancer factor 1 (LEF-1) but not of the highly related T-cell factor 1 (TCF-1). Pendulin is the mouse homologue of human Rch1/Srp1α/importin-α. In contrast to a low-level of expression of mSrpl and pendulin in all tissues examined, mouse pendulin is highly expressed in spleen, thymus and heart. Pendulin may perform additional or unique functions in tissues that express high levels of this protein. Increased expression of pendulin in lymph nodes of the mouse asthma model was observed. The invention provides a method for treatment and/or prevention of an immune related response, more preferably asthma and related auto-immune and inflammatory diseases, comprising modulating expression of pendulin, more preferably increasing expression of pendulin.

The invention provides a method for modulating an immune response comprising modulating a gene(s) involved in signal transduction cascades leading to the production of cytokines and/or chemokines and/or growth factors pertaining to an immune response. Cytokines are primarily involved in signaling between cells of the immune system (e.g IL-4, IL-6, IL-8, IL-17 and Il-18). Chemokines are defined primarily as those compounds that draw cells and other factors to sites of injury in the body (e.g human GRO-β, Human IP-10). Growth factors promote cell division and proliferation of certain cell types (e.g human transforming growth factor β-1 etc).

The invention provides a method for modulating an immune response comprising modulating a gene, wherein said gene is involved in sensory nerve activation involved in an immune response. More preferably the immune response is an inflammatory response. Chloride channels appear to be involved in neuronal excitability. Dorsal root ganglia contain sensory nerve bodies that are involved in neurogenic inflammation which contributes to allergic inflammation and pain (inflammatory hyperalgesia). Interference with these chloride channels blockade of hCaCC1 (or gob-5) and/or hCaCC3 (or the marine homologue) by selective antagonists can limit neurogenic inflammation in asthma and other diseases with a neurogenic inflammatory component. Furthermore, cough, which is a prominent symptom of asthma, is believed to be a result of sensory nerve activation. The invention provides a method for the treatment of immune related responses comprising providing blockade of hCaCC1 (or gob-5) and/or hCaCC3 (or the murine homologue) by selective antagonists.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example blockade of hCaCC1 (or gob-5) by a selective antagonist inhibits mast-cell activation and can be used in diseases in which mast cells play an important role such as all allergic diseases (rhinitis, atopic dermatitis, asthma, urticaria) and auto-immune diseases (i.e. multiple sclerosis). Blockade of hCaCC1 (or gob-5) and/or hCaCC3 inhibits the excitability of sensory neurons and thereby prevents or decreases (1) the neurogenic component of inflammatory responses, (2) hyperalgesia during inflammatory responses and (3) cough due to airway inflammation. Activation of receptors (PAR2) by serine proteases is sensitive to inhibition by APLP2 or its KPI domain and treatment with APLP2 or its KPI domain or induction of APLP2 expression is effective in the treatment of bronchoconstriction induced by activation of sensory nerves.

The invention provides a method for modulating an immune response comprising modulating a gene wherein said gene modulates a Th1 (by way of example but not limitation auto-immune diseases) and/or Th2 (by way of example but not limitation inflammatory diseases) mediated immune response. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance for example membrane C-type lectin like homologue (EST AA914211: signature sequence OtS1-B7). C-type (Ca²⁺-dependent) lectins represent an important recognition mechanism for oligosaccharides at cell surfaces, attached to circulating proteins and in the extra-cellular matrix. Binding of specific sugar structures by these lectins mediates biological events such as cell-cell adhesion, serum glycoprotein turnover and innate immune responses to potential pathogens. These proteins contain carbohydrate-recognition domains (CRDs) that mediate sugar binding. C-type lectins also contain a Ca²⁺ binding site. C-type lectins have been demonstrated to be present in antigen-presenting cells such as macrophages and dendritic cells. Interestingly, alveolar macrophages have been demonstrated to phagocytose allergens via an undefined C-type lectin leading to the induction of iNOS and subsequent generation of NO by alveolar macrophages. The NO generated by these macrophages may drive T-cell differentiation into the Th2 pathway by inhibition of Th1 responses. The invention provides a method for the treatment and/or prevention of an immune related response comprising providing the targeting of an antigen to this C-type lectin. This induces a Th2 dominated immune response and is effective in the treatment of Th1 mediated diseases such as auto-immune diseases.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example a protein inhibitor of neuronal nitric oxide synthase (mPIN) (signature sequence R1-OS-B1-B1). Nitric oxide (NO) can be produced by several nitric oxide synthase enzymes (nNOS, iNOS and eNOS). Murine PIN is a cytoplasmic protein and is a selective inhibitor of neuronal nitric oxide synthase (nNOS). The human homologue appears to be dynein light chain 1 hdlc1). NO has been implicated in several diseases including asthma and other inflammatory diseases. Interestingly, nNOS is located on chromosome 12q that has been linked to asthma. The invention shows a down-regulation of mPIN mRNA in lymph nodes of a mouse asthma model. NO negatively regulates type-1 T-helper lymphocyte (Thl) development. Likewise, NO may tip the balance between Th1 and Th2 cells in favor of Th2 responses. The invention provides a method for the treatment and/or prevention of an immune related response, more particular Th2-mediated immune responses such as allergy and asthma comprising modulating PIN expression, more preferably decreasing expression which leads to increased NO release and facilitation of Th2-mediated immune responses such as allergy and asthma. The invention provides a method for the treatment and/or prevention of an immune related response, comprising blockade of PIN activity which is beneficial in Th1 mediated diseases such as auto-immunity by increasing regulatory Th2 cells. Treatment with PIN is beneficial in Th2 mediated responses such as asthma and allergy by increasing regulatory Th1 cells. Besides a role of PIN in the regulation T-cells, it plays a role in airway hyper-responsiveness. Neuronal NOS but not iNOS nor eNOS has been demonstrated to be crucial for baseline- and antigen-induced airway hyperresponsiveness in mice. Expression of nNOS but not eNOS nor iNOS in airway epithelial cells of our mouse model of allergic asthma is demonstrated. The invention provides a method for the treatment and/or prevention of an immune related response, comprising modulating nNOS and PIN, more preferably up-regulating nNOS in airway epithelial cells and down-regulating PIN. Up-regulation of nNOS in airway epithelial cells and a down-regulation of PIN can strongly potentiate the production of NO or its metabolites. The invention provides a method for the treatment and/or prevention of an immune related response, comprising modulating expression of PIN, more preferably increasing expression of PIN which inhibits NO production by nNOS and inhibits airway hyperresponsiveness in asthma and related respiratory diseases associated with hyperresponsiveness such as COPD.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma for example cathepsin B (signature sequence: OtS2-F2). Exogenous antigens are processed by lysosomal proteases within antigen-presenting cells to create antigenic peptides which are loaded into MHC class II molecules and expressed on the cell-surface to CD4⁺ T-lymphocytes. Enzymes such as aspartate roteases (e.g. cathepsin D and E) and cysteine proteases (e.g. cathepsin B, L and S) are proposed to be involved in this process. Interestingly, cathepsin B appears to be involved in the generation of Th2 dominated immune responses to ovalbumin and to a Leishmania infection in BALB/c mice. The invention provides a method for the treatment and/or prevention of an immune related response, comprising providing inhibition of the activity of cathepsin B by inhibitors. This inhibits allergic asthma and related allergic and Th2-mediated inflammatory responses.

Furthermore the invention provides a method for the treatment and/or prevention of an immune related response, comprising providing targeting of antigen to LM which will induce a Th2 dominated immune response and is effective in the treatment of Th1 mediated diseases such as auto-immune diseases. The invention provides a method for the treatment and/or prevention of an immune related response, comprising modulating Ly-GDI and/or Cdc42-GAP, more preferably inducing the expression of these proteins. Modulating Ly-GDI and/or Cdc42-GAP, or inducing the expression of these proteins induces T-helper lymphocyte type-2 responses and is effective in the treatment of Th1-lymphocyte mediated diseases like auto-immune diseases.

The invention provides a nucleic acid library comprising nucleic acid or functional fragments, derivatives or analogues thereof comprising genes as listed in table 1, 2 or 3 which are implicated in oxidative stress responses and/or programmed cell death (PCD) (i.e cellular apoptosis). The invention provides a method for treatment of an immune response wherein said nucleic acid is involved in the generation of anti-oxidants or free radicals. An ‘antioxidant’ or free radical scavenger is an enzyme that prevents build up of reactive oxygen species (ROS) in cells. In general anti-oxidants prevent tissue damage by oxidative stress. Free ‘radical generator’ is a enzyme that is involved in the generation of ROS.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma for example clusterin/Apolipoproteine J/sulphated glycoprotein 2 (signature sequence OtS2-B12). Clusterin is a 75-80 kDa disulphide-linked heterodimeric secreted glycoprotein. It is encoded by a single gene and the translated product is internally cleaved to produce its α and β subunits prior to secretion from the cell. It is ubiquitously expressed. There is extensive evidence of a correlation between clusterin expression and diseases e.g. Alzheimer, glioma's or pathological stress. Many functions have been ascribed to clusterin such as controlling cell-cell and cell-substratum interactions; regulating apoptosis; transporting lipids; regulating complement and a general chaperone/heat-shock protein function.

The invention provides a method for the treatment and/or prevention of an immune related response, comprising modulating clusterin, more preferably increasing the expression of clusterin, which will inhibit allergic asthma and related allergic and inflammatory diseases.

Moreover, anti-oxidants may inhibit the expression of genes regulated by the “redox status” within inflammatory cells, such as the ras pathway. Oxidative stress also appears to be involved in the activation of the CD4-associated protein tyrosine kinase p56^{lck}. P56^{lck} is an important protein in the activation of CD4⁺ T-lymphocytes. Oxidative stress is increased in patients with asthma and chronic obstructive pulmonary disease (COPD) and it is possible that reactive oxygen species contribute to its pathophysiology. Likewise, antioxidants might be of use in the therapy of these respiratory diseases. Oxidative stress has also been shown to regulate the cellular glucocorticoid responsiveness. A decreased sensitivity to glucocorticoids has been observed in patients with allergic asthma leading to treatment with either high-doses of glucocorticoids or inappropriate treatment. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention provides various anti-oxidant proteins down-regulated upon OVA-challenge in the mouse asthma model e.g Selenoprotein P (signature sequence: R1-OS-B1-H1), Gluthation-S-transferase mu2 (signature sequence: OtS2-E6), Ferritine (signature sequence: R1-OS-B1-O5), Anti-oxidant protein 2 (signature sequence: OtS2-A6).

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma for example selenium. Selenium is an essential trace element that is incorporated as selenocysteine into the primary structure of selenoprote ins. There are at least 10 animal selenoproteins. Animal studies have demonstrated a role for selenium in oxidant defense, thyroid hormone metabolism, and defense against viral infections. Selenoproteins presumably mediate these biologic effects. Most of the human selenoproteins are members of the gluthatione peroxidase or iodothyronine deiodinase families. Selenoprotein P (SEPP1) is not a member of these families. It is an extracellular glycoprotein that is present in several isoforms and is the only selenoprotein known to contain multiple selenocysteine residues. It is a heparin-binding protein that appears to be associated with endothelial ceus and has been implicated as an oxidant defense in the extracellular space. There is evidence that several isoforms of the protein exist, likely products of the same gene. Human selenoprotein has been mapped to chromosome 5q31. Interestingly, many studies have demonstrated a linkage between chromosome 5q and allergy, asthma and airway hyperreactivity. There is considerable evidence that oxidative stress is increased in patients with chronic obstructive pulmonary disease (COPD) and that reactive oxygen species contribute to its pathophysiology. Likewise, it has been postulated that antioxidants might be of use in the therapy of COPD. Selenoprotein P may be useful as a therapeutic protein in diseases that are associated with increased oxidative stress such as COPD, asthma and other inflammatory diseases. It was observed that mRNA levels of selenoprotein P are decreased in lymph node tissue of a mouse asthma model. Selenium and selenoproteins have been shown to play a role in the function of granulocytes and lymphocytes. The invention provides a method for the treatment and/or prevention of an immune related response, comprising maodulating selenoprotein P.

The invention provides a method for modulating an immune related response, comprising modulating the generation of anti-oxidants or free radicals. Treatment with anti-oxidant proteins (e.g by inhalation) or induction of the expression of these proteins and/or suppression of free radical generators in airway tissue can be used to treat allergic inflammation or related inflammatory diseases or diseases associated with increased oxidative stress such as asthma and COPD. Treatment with anti-oxidant proteins or induction of the expression of these proteins in airway tissue together with glucocorticoid treatment can limit the dose of glucocorticoids required for a therapeutic effect in patients with allergic asthma and other chronic inflammatory diseases associated with glucocorticoid insensitivity.

The invention provides a substance such as a proteinaceous ubstance capable of modulating a gene comprising a nucleic acid at least functionally table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma for example cytochrome P-450 naphtalene hydroxylase (CYP2F2) enzymes (signature sequence R1-OS-B1-A1). CYP2F2 are a superfamily of more than 160 known members that play a major role in the metabolism of numerous physiological substrates and a wide array of xenobiotics including drugs, chemical carcinogens, insecticides, petroleum products, and other environmental pollutants. Oxidative metabolism catalyzed by cytochrome P450s can result in detoxification. In some instances it results in metabolic activation of a chemical to cytotoxic and/or carcinogenic forms. Although the liver is the primary organ for drug metabolism, extrahepatic tissues such as lung, kidney and intestine, also play an important role in detoxification or biotransformation of xenobiotics. Each tissue has a unique P450 isozyme distribution and regulatory mechanism for cytochrome P450 gene expression. Currently, the members of the CYP2F gene subfamily that are selectively expressed in lung tissues consist of human CYP2F1 and mouse CYP2F2 and CYP2F3. Human CYP2F1 bioactivates 3-methylindole, while mouse CYP2F2 bioactivates naphtalene. Mouse CYP2F3 catalyzes the dehydrogenation of 3-methylindole but not its hydroxylation. Murine CYP2F2 is expressed in lung tissue as well as in liver. In the lung, it plays an important role in the metabolic activation of substrates that cause lung injury. CYP2F2 is involved in the hydroxylation of naphtalene and it specifically catalyses the production of a very reactive and potentially toxic intermediate, the 2R, 2S arene oxide, that is associated with necrosis of unciliated bronchiolar epithelial cells or CLARA cells in lung. Several P450 enzymes with epoxygenase activity have also been shown to be involved in the metabolism of arachidonic acid into biologically active eicosanoids. Based on the bioactivation of naphtalene, we anticipate that CYP2F enzymes also displays epoxygenase activity. The epoxygenase pathway leads to the formation of four regio-isomeric epoxy-eicosatrienoic acids (EETs): 14,15-EET, 11,12-EET, 8,9-EET and 5,6-EET. From these epoxides, other lipid mediators can be generated such as 14,15-DHET, 11,12-DHET, 8,9-DHET, 5,6-DHET and 5,6-epoxy prostaglandin El. Some of these epoxides have been shown to induce vasorelaxation. 5,6-EET and 11,12-EET have also been shown to modulate tracheal chloride-channel activity and induce airway smooth muscle relaxation. Epoxides generated through CYP2F may therefore protect against excessive bronchoconstriction and may be involved in airway hyperreactivity in asthma and other respiratory diseases. Epoxygenase metabolites have also been shown to have anti-inflammatory activities such as inhibition of leukocyte adhesion to the vascular wall and inhibition of IκB kinase thereby preventing the activation of NF-κB. Cytochrome P-450 naphtalene hydroxylase (CYP2F2). A strong (>10-fold) down-regulation of cytochrome P450 (CYP2F2) mRNA in a mouse asthma model in the lymph nodes of “asthmatic” (OVA-challenged) compared to “healthy” (saline-challenged) mice was observed. The invention provides a method for the treatment and/or prevention of an immune related response, comprising modulating the expression of CYP2F, more preferably increasing expression of CYP2F in airway tissue and/or by preventing its down-regulation. This inhibits airway hyperresponsiveness and excessive bronchoconstriction and can be used to treat allergic asthma and other respiratory diseases associated with hyperresponsiveness such as COPD. The invention provides a method for the treatment and/or prevention of an immune related response, comprising providing local treatment (inhalation) with CYP2F metabolites of arachidonic acid, in particular 11,12-EET, which inhibits airway inflammation for treatment of allergic asthma and other respiratory inflammatory diseases such as COPD. The invention provides for a method of treatment and/or prevention of an immune related response, comprising modulating the enzymatic activity of CYF2F, more preferably stimulating the enzymatic activity of CYF2F by an allosteric stimulator which increases the generation of epoxides and likewise inhibits airway hyperresponsiveness and airway inflammation. Stimulation of the enzymatic activity of CYF2F by an allosteric stimulator is effective in the treatment of allergic asthma and other respiratory diseases such as COPD.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example four families of structurally related heat-shock proteins are distinguished based on their molecular weights: Hsp90, Hsp70, Hsp60 and small Hsp's. By definition, Hsp expression is elevated in cells undergoing stress, such as those in damaged or inflamed tissue. Conditions as diverse as a rise in temperature, hypoxia, irradiation, infection and exposure to toxic chemicals can all result in increased Hsp expression. seat-shock cognate protein (Hsc)73 is a constitutively expressed member of the Hsp70 family. Hsc73 is expressed in the cytosol but is also present in lysosomes. Hsc73 plays a role in binding and protecting peptides from extensive degradation and facilitating the kinetics of peptide transfer to MHC class II molecules. Hsc73 is also present in dendritic cell-derived exosomes which have been shown to elicit potent T-cell dependent immune responses in mice. Moreover, a receptor for Hsp70 proteins is present on the surface of macrophages and dendritic cells and Hsp70 can induce macrophages to activate T-cells independently of antigen. Thus, Hsc73 appears to be involved in antigen-presentation and T-cell activation. Administration of antigen or antigenic peptides together with Hsp70 proteins has been shown to generate CD8⁺ T-lymphocyte responses when administered to laboratory animals. Moreover, Hsp70 is involved in cross-priming of CD8⁺ cells by APC upon antigen processing. Recently, Hsp70 has also been shown to be involved in the induction of regulatory T-cells. Hsc73 (signature sequence: OtS2-H2) may also be involved in the induction of inducible nitric oxide synthase (iNOS) by LPS or cytokines via an effect on p38 mitogen-activated protein (MAP) kinase. In agreement herewith, the selective hsc73 inhibitor deoxyspergualin inhibits the induction of iNOS by cytokine- or endotoxin-activated macrophages. NO has been shown to inhibit the generation of Th1 lymphocytes thereby tipping the balance towards Th2 immune responses. In airway epithelial cells, Hsp70 has been shown to have potent anti-inflammatory effects by stabilization of Iκ Bα through preventing the activation of IκB kinase leading to inhibition of NF-κB activation and down-stream gene transcription. In airway epithelial cells, increased Hsp70 expression suppressed cytokine-induced expression of pro-inflammatory cytokines IL8 and TNFα.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against Hsc73 . This inhibits the generation of NO by APC's and thereby limits a Th2 dominated immune response by increasing Th1 immunity. This treatment is effective in the treatment of allergic asthma and related allergic and inflammatory responses.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example, the invention provides for a method of treatment and/or prevention of an immune related response, more particularly allergic inflammation or related inflammatory diseases (eg. COPD) comprising modulating, more preferably up-regulating the expression of Hsc73 leading to induction and/or elevation of the expression of Hsc73 protein in airway epithelial cells.

The invention provides a method for treatment of an immune response comprising providing an antagonist of antigen processing and presentation. ‘Antagonist’ herein refers to a molecule that bears sufficient structural similarity to a second molecule to compete with that molecule for binding sites on a third molecule, such as for example an antibody. An ‘antibody’ herein refers to a protein produced by lymphoid cells in response to foreign substances (antigens) and capable of coupling specificauly with it's homologous antigen (the one that stimulated the immune response) or with substances that are chemically very similar to that antigen. Antibody herein refers to both polyclonal and monoclonal antibodies.

The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance by way of example the invention provides nucleic acids as listed in table 1, 2 or 3 which are involved in antigen processing and presentation MHC-II (signature sequence: StO1-B5), H2-Oa (MHC-II: signature sequence: SvO2-1-A4), EST: Clathrin (signature sequence: SvO2-1-D4), Aspartyl aminopeptidase (signature sequence: StOl-c1), Cathepsin B (signature sequence: OtS2-F2), Breast heat shock 73 protein (signature sequence: OtS2-H2), EST: C-type lectin (signature sequence: OtS1-B7), Ubiquitin-specific protease (signature sequence: R1-OSB1-A2), Ubiquitin/60s (signature sequence: SVO2-1-C12) and Lysozyme M (OtS2-B1). Antigen-presenting cells play an important role in the differentiation of CD4⁺ and CD8⁺ T-lymaphocytes into particular subsets (Type-1, Type-2, Type-3 or regulatory types) and are important for the generation of either a detrimental or a beneficial immune response to antigens.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example Phospholipase Cγ2 (PLCγ2). (signature sequence: SvO2-1-A8). PLCγ2 unlike PLCγ1 which is expressed in many cell-types, PLCγ2 is only expressed in hematopoietic cells (e.g. B-lymphocytes, NK-cells, platelets, granulocytes, monocytes/macrophages and mast cells). PLCγ2 is a cell signaling molecule with many regulatory domains e.g. SH2, SH3, pH domains. It catalyzes the hydrolysis of phosphatidyl-inositol 4,5-biphosphate to yield the second messengers, IP3 and DAG, PLCγ2 has been shown to be involved in production of reactive oxygen intermediates by neutrophils. In addition to PLCγ1, PLOCγ2 is activated upon triggering of mast cells via Fce RI. The promoter region of PLCγ2 has Sp1, NF1, AP2, SRE, EBF and CACCC box consensus sites. In B-cells, mRNA expression of PLCγ2 is enhanced by serum, TPA, retinoic acid and 5-azacytidine. The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against PLCγ2 or a proteinaceous substance comprising PLCγ2 is effective in the treatment of allergic asthma and related allergic and inflammatory diseases.

The invention provides for a method of treatment and/or prevention of an immune related response, comprising providing an antagonist(s) directed against a proteinaceous substance derived from a nucleic acid sequence at least functionally equivalent to a nucleie acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example APLP2 C31 (signature sequence: SvO2-1-B7) peptide is involved in cell death (apoptosis). Apoptosis or cell-death is an important mechanism to limit immune reactions. The cytoplasmic domain of APLP2 containing the “NPTY” motif is involved in T-lymphocyte activation upon phosphorylation of the tyrosine (Y) residue leading to Shc binding. The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against APLP2, more specifically the cytoplasmic domain of APLP2 containing the “NPTY” motif. This prevents the Ras-pathway of T-lymphocyte activation and inhibits an immune response and is effective in the treatment of allergic asthma and related allergic and inflammatory diseases. The invention provides for a method of treatment and/or prevention of an immune related response, comprising providing an antagonist(s) directed against “VEVD” and “NPTY” motif inhibits unwanted cell death mediated by this pathway and is effective in the treatment of allergic asthma and related allergic and inflammatory diseases. The invention provides for a method of treatment and/or prevention of an immune related response, comprising providing an antagonist(s) directed against a proteinaceous substance derived from a nucleic-acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing inhibition of the generation of the C-terminal 31 mino acid APLP2 peptide by caspases and/or proteases encoded by the nucleic acid of table 1, 2 or 3 which inhibits unwanted cell death mediated by this pathway.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against hCaCC1 (or gob-5) (signature sequence: R1-SO-R1-C11) which inhibits mast cell activation and can be used in the treatment of immune diseases in which mast cells play an important role such as all allergic diseases (rhinitis, atopic dermatitis, asthma, urticaria) and auto-immune diseases (i.e. multiple sclerosis).

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1, 2 or 3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against Hsc78. This inhibits the generation of NO by APC's and thereby limits a Th2 dominated immune response by increasing Th1 immunity. This treatment is effective in the treatment of allergic asthma and related allergic and inflammatory responses.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1, 2 or 3 and use of said substance for the production of an antagonist against said substance. The invention further provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against LR8 which inhibits allergic asthma and related allergic and inflammatory diseases.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance. The invention further provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma. More preferably a method of treatment and/or prevention of an immune related response, more preferably allergic asthma and related allergic and inflammatory diseases, comprising providing an antagonist(s) directed against one or more up-regulated genes as listed in table 1, 2 or 3 or the corresponding proteinaceous substances.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance. The invention further provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against LY-GDI (signature sequence: SVO2-1-D8) and/or Cdc42-GAP (signature sequence, R1-SO-R1-A12) which inhibits T-helper lymphocyte type-2 (Th2) responses and is effective in the treatment of allergic asthma and related allergic and Th2-mediated inflammatory diseases.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least finctionally equivalent to a nucleic acid identifiable by a signature sequence as shown in table 1-3 and use of said substance for the production of an antagonist against said substance. The invention farther provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma. For example the invention provides for a method of treatment and/or prevention of an immune related response, comprising providing an antagonist(s) directed against C-type lectin (signature sequence: Ot-S2-B7) which inhibits antigen presentation and skewing towards a Th2 dominated immune response. This blockade is effective in the treatment of allergic asthma and related allergic and inflammatory diseases.

The invention provides a method for modulating an immune response wherein said gene modulates CD8+T-lymphocyte responses. Also provided is a gene or gene product capable of inducing a specific regulatory CD4⁺ and/or CD8⁺ T-lymphocyte response that inhibits Th2 dominated allergic responses. The invention provides a method for modulating an immune response wherein said gene modulates CD4⁺ T-lymphocyte responses. The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance. The invention further provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma for example Ubiquitin-speciiic protease (UBP43)(signature sequence R1-OS-B1-A2). UBP43 belongs to a family of ubiquitin-specific proteases (UBP) and has a molecular mass of 43 kDa. Protein ubiquitination has been implicated in many important cellular events. The human homologue of this protein is ISG43. In wild-type adult mice, UBP43 is highly expressed in thymus and peritoneal macrophages. Furthermore, it is expressed in cell-lines of the monocytic lineage and its expression is regulated during cytokine-induced monocytic cell differentiation. Over expression of UBP43 has been shown to block cytokine-induced terminal differentiation of the monocytic cell-line M1. Down-regulation of UBP43 mRNA in lymph nodes of a mouse asthma model was observed. The invention provides for a method of treatment and/or prevention of an immune related response, comprising modulating the expression of UBP43, more preferably increasing the expression of UBP43 in APC's which prevents allergic asthma and related respiratory disease by increasing the generation of regulatory CD8⁺ T-lymphocytes. The proteasome is involved in the generation of MHC class-I peptides by proteases.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance. The invention further provides the use of said antagonist such as an antibody directed against a proteinaceous substance derived from at least a nucleic acid as shown table 1-3 for the production of a medicament for the treatment of an immune response observed with airway hyperresponsiveness and/or bronchoalveolar manifestations of asthma. For example the invention provides for a method of treatment and/or prevention of an immune related response, comprising providing inhibition of peptide loading into MHC class-I molecules by proteases encoded by the nucleic acid as outlined in table 1, 2 or 3, which inhibits the generation of CD8⁺ T-lymphocyte responses (i.e T-lymphocyte costimulation). Airway wall remodeling is an established pathological feature of asthma but its causes are not well understood. One cytokine of potential relevance is transforming growth factor-betal (TGF-beta 1). In patients with asthma, matrix-associated TGF-beta 1 is likely to be bound at least in part to decorin (signature sequence: R1-OS-B1-C5). This interaction may provide a reservoir of TGF-beta 1 that can be released in an active form in response to appropriate stimuli. Decorin is also a natural inhibitor of TGF-beta and has been shown to restore T-lymphocyte responses to mycobacteria. The invention provides for a method of treatment and/or prevention of an immune related response, comprising modulating the expression of decorin, preferably increasing the expression of decorin. Increased expression of decorin in airway tissue and/or treatment (inhalation) with decorin inhibits the effects on TGF-beta on airway tissue remodeling and is effective in the treatment of immune related responses.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance for example the invention provides for a method of treatment and/or prevention of an immune related response, comprising providing immunotherapy using Hsc73, alone or together with antigen/allergen. An allergen herein is defined as a substance inducing hypersensitivity. Immunotherapy using Hsc73, alone or together with antigen/allergen induces a specific regulatory CD4⁺ or CD8⁺ T-lymphocyte response that inhibits Th2 dominated allergic responses.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance for example the invention provides a method for modulating an immune response of an individual wherein said gene encodes a gene product capable of modulating an immune response. A gene product herein refers the mRNA and the polyp eptide chain translated from an mRNA molecule, which in turn is transcribed from a gene; if the RNA transcript is not translated (e.g rRNA, tRNA) the RNA molecule represents the gene product. The gene product herein can refer to any proteinaceous substance. A proteinaceous substance can refer to any molecule comprising amino acid and/or peptide or protein.

The invention provides alleles of the polypeptide(s) encoded by nucleic acid sequences of this invention. As used herein, an ‘allele’ or ‘allelic sequence’ is an alternative form of the polypeptides described above. Alleles result from a mutation [eg. a change in the nucleic acid sequence, and generally produce altered mRNA or polypeptide whose structure or function may or may not be altered]. Any given polypeptide may have none, or more alelic forms. Common allelic changes that give rise to alleles are generally ascribed to natural deletions, additions or substitutions of amino acids. Each of these types of changes may occur alone, or in combination with the others, one or more times in a given sequence. Deliberate amino acid substitution may be made on the basis of similarity in polarity, charge, solubility, hydrophobicity, and/or the amphipathetic nature of the residues as long as the biological activity of the polypeptide is retained. Altered nucleic acid sequences of this invention include deletions, insertions, substitutions of different nucleotides resulting in the polynucleotides that encode the same or are functionally equivalent. A ‘deletion’ is defined as a change in either nucleotide or amino acid sequence in which one or more nucleotides or amino acid residues, respectively, are absent. An ‘insertion’ or ‘addition’ is that change in nucleotide or amino acid sequence which has resulted in the addition of one or more nucleotides or amino acid residues, respectively, as compared to the naturally occurring polypeptide(s). A ‘substitution’ results from the replacement of one or more nucleotides or amino acids by different nucleotides or amino acids, respectively. The invention includes variants of the polypeptide. A ‘variant’ of a polypeptide is defined as an amino acid sequence that is different by one or more amino acid ‘substitutions’. A variant may have ‘conservative’ changes, wherein a substituted amino acid has similar structural or chemical properties e.g. replacement of leucine with isoleucine. More rarely a variant may have ‘non-conservative’ changes (eg replacement of a glycine with a tryptophan). Similar minor variations may also include amino acid deletions or insertions, or both. Guidance in determining which and how many amino acid residues may be substituted, inserted or deleted, without abolishing biological or immunological activity may be found using computer programs well known in the art, for example, DNAStar software.

The invention provides a method modulating an immune response wherein said immune response comprises airway hyperresponsiveness and/or bronchoalveolar manifestations of asthma.

The invention provides a method modulating an immune response wherein said gene is modulated by transducing a cell of said individual. Methods to transduce cells are known in the art. Target cells can be transduced with a nucleic acid delivery vehicle comprising at least one nucleic acid the subject of the invention. A ‘gene delivery vehicle’ herein is used as a term for a recombinant virus particle or the nucleic acid within such a particle, or the vector itself, wherein the vector comprises the nucleic acid to be delivered to the target cell(s) and is further provided with a means to enter said cell(s). This cell(s) can be used for drug screening and drug discovery.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance, for example the invention provides a substance capable of modulating a gene comprising a nucleic acid at least fuictionally equivalent to a nucleic acid identifiable by a signature sequence as shown in table 1, 2 or 3.

A substance herein refers to any material entity capable of modulating a gene the subject of the invention, for example an ‘entity’ can be a molecule wherein said molecule is a chemical compound. The substance can also be an ‘antigen’ a foreign invader comprising a protein or protein attached moiety. The substance can also be of proteinaceous origin comprising amino acid and/or peptide or protein.

The invention provides a medicament comprising a substance capable of modulating a gene(s) the subject of the invention. A preferred embodiment is a medicament which is a pharmaceutical. Suitable pharmaceutical compositions are known.

The invention provides the use of a substance for the production of a medicament for the treatment of an immune response observed with airway hyperresponsiveness and/or bronchoalveolar manifestations of asthma.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance, for example the invention provides the use of a proteinaceous substance derived from a nucleic acid at least functionally equivalent to a nucleic acid identifiable by a signature sequence as shown in table 1, 2 or 3 for the production of an antagonist against said substance. ‘Antagonist’ herein refers to a molecule that bears sufficient structural similarity to a second molecule to compete with that molecule for binding sites on a third molecule, for example an antibody.

The invention provides the use of a proteinaceous substance derived from a nucleic acid at least functionally equivalent to a nucleic acid identifiable by a signature sequence as shown in table 1, 2 or 3 for the production of an antagonist against said substance, wherein said antagonist is an antibody or functional equivalent thereof. An ‘antibody’ herein refers to a protein produced by cells in response to foreign substances (antigens) and capable of coupling specifically with it's homologous antigen (the one that stimulated the immune response) or with substances that are chemically very similar to that antigen. Antibody herein refers to both polyclonal and monoclonal antibodies.

The invention provides for a method of treatment and/or prevention of an immune related response, comprising providing an antagonist(s) directed against a proteinaceous substance derived from a nucleic acid sequence at least functionally equivalent to a nucleic acid identifiable by a signature sequence as shown in table 1, 2 or 3. The invention provides a substance such as a proteinaceous substance capable of modulating a gene comprising a nucleic acid at least functionally equivalent to a nucleic acid identifiable by a signature sequence as shown in table 1-3 and use of said substance for the production of an antagonist against said substance for example the invention provides an antagonist directed against a proteinaceous substance derived from a nucleic acid at least functionally equivalent to a nucleic acid identifiable by a signature sequence as shown in table 1, 2 or 3. ‘Functionally equivalent’ herein means that the subject signature sequence can vary from the reference sequence by one or more substitutions, deletions, or additions, the net effect of which will not result in a functional dissimilarity between the two sequences.

The invention provides an antagonist comprising an antibody or functional equivalent thereof An antibody or functional equivalent thereof can refer to synthetic molecules (i.e antibodies derived by chemical synthesis) and encompasses all molecules capable of coupling with proteinaceous substance(s) derived from nucleic acid of the invention. Proteinaceous substance herein can refer to an entity derived from said nucleic acids the subject of the invention capable of modulating an immune response.

The invention provides a medicament comprising an antagonist. The invention provides the use of an antagonist for the production of a medicament for the treatment of an immune response observed with airway hyperresponsiveness andjor bronchoalveolar manifestations of asthma.

The invention provides for use of an antagonist for the production of a medicament for the treatment of an immune response observed with airway hyperresponsiveness and/or bronchoalveolar manifestations of asthma.

The invention further provides a diagnostic kit for screening for an immune response comprising providing a nucleic acid the subject of the invention. Methods of screening are known in the art. These procedures include, but are not limited to DNA-DNA, DNA-RNA hybridisation. The form of such quantitative methods may include, Southern or Northern analysis, dot/slot blot or other membrane based technologies; PCR technologies such as DNA Chip, TaqmanS, NASBA, SDA, TMA, in-situ-hybridisation, protein bioassay or immunoassay techniques ELISA, IFA, proteomic and metabolic technologies.

### EXAMPLES

### Example 1

### Development of Murine Model of Allergic Asthma

Due to the limitations of experimental studies in patients with allergic asthma a murine model with immunologic and pathophysiologic features reminiscent of allergic asthma was developed (Oosterhout A J (1998): Am J Respir Cell Mol Biol; 19:826-35). There are several advantages to using a murine model compared to using tissues obtained from asthma patients such as (i) availability of isolated tissues or cells (ii) genetic homogeneity, (iii) identical age, (iv) well-controlled environment (food, specified pathogens, climate), and (v) ability to do time-series experiments (i.e induction vs effector phase). In this model, Balb/c mice are sensitized with ovalbumin (OVA) and repeatedly challenged by inhalation of OVA aerosol. This model is characterized by the presence of OVA-specifc IgE antibodies in serum, airway eosinophilia and non-specific hyperresponsiveness concomitant with the appearance of Th2-like cells in lung tissue and lung draining (thoracic) lymph nodes.

### Example 2

### Representational Difference Analysis (RDA)

Representational Difference Analysis of cDNA's (RDA) was employed to identify novel key regulatory molecules involved in the initiation and/or progression and/or suppression and/or repression of asthma symptoms. RDA analysis was performed according to previously defined methods [Groot and van Oost (1998). Nucleic Acids Res: 26:4476-81][Welford et al., (1998): Nucleic Acids Res 1998; 26:3059-65][Geng et al., (1998): Biotechniques 25:434-8]. Gene expression between lung-draining lymph nodes (containing amongst others dendritic cells, macrophages, B- and T-lymphocytes, mast cells) obtained from “healthy” control animals and those obtained from “asthmatic” mice that display airway manifestations of asthma such as airway hyperresponsiveness and bronchoalveolar eosinophilia were compared. Balb/c mice were intraperitoneally sensitized with ovalbumin and later on repeated challenged by inhalation of saline aerosol (control or “healthy” animals) or ovalbumin aerosol (“asthmatic”). Lymph nodes were isolated at 6 hours after the last challenge. Using RDA differentially expressed gene fragments were identified. Up-regulated genes are those that are expressed at higher levels in asthmatic tissue compared to healthy tissue. Vice versa, down-regulated genes are those that are expressed at lower levels in asthmatic tissue compared to healthy tissue. NCBI (National center of biotechnology information) BLAST searches with the differentially expressed gene fragments against publicly available databases revealed significant alignment with either known genes (human or mouse), with expressed sequence tags (EST's) or in some cases did not reveal a significant alignment or an incomplete alignment (unknown genes). The identified differentially expressed genes are listed in Table 1.

### Example 3

### Microarray Experiment

Detection of dierentially expressed genes in “asthmatic” mice compared with “healthy” control animals was performed using representational differences analysis coupled to microarray hybridization methods as described previously [Welford et al., (1998). Nucleic Acids Res: 26:3059-65]. Unique differentially expressed genes (tethered nucleic acid: target) obtained from the RDA experiment (example 2) were amplified by PCR using M13 primers, precipitated and spotted (arrayed in duplicate) onto chemicaUy-modified glass slides (Corning) using a robotic printing device. Messenger RNA obtained from both lymph nodes of “healthy” and from “asthmatic” mice was transcribed into double-stranded cDNA and amplicons were generated. Amplicons were subsequently fluorescently labeled with either cyanine 3 (Cy3-ULS) or cyanine 5 (Cy5-ULS) dyes (i.e one mRNA population (probe: free nucleic acid) was labeled with cyanine 3 (Cy3-ULS) and the other with cyanine 5 (Cy5-ULS)). The labeled probes (free nucleic acids) were then mixed and hybridized simultaneously to a microarray. The microarray was hybridized with both the Cy3 and Cy5 labeled probes in order to determine the expression ratio between both samples. After hybridization, the fluorescence pattern of each microarray was recorded for the Cy3 and Cy5 fluorescent dyes. Detailed statistical analyses were applied in order to determine the minimal significant ratio in each experiment. Clones that exhibited differential fluorescence were identified. In table 1, the expression ratio (“asthma”: “healthy”) is given.

### Example 4

### Virtual Northern Blot

Messenger RNA obtained from lymph nodes of “healthy” and “asthmatic” mice was transcribed into double-stranded eDNA and amplicons were generated. Using agarose gel electrophoresis, different amounts of amplicons were run and subsequently blotted onto Hybond flter membrane. Specific and individual gene fragments obtained by RDA from the lymph nodes of “healthy” and “asthmatic” mice were subcloned and subsequently amplified using-M13 primers and fluoreseently labeled (by random primer labeling). Labeled gene fragments were hybridized on the filter membrane containing the blotted amplicons and analyzed by a fluor-imager. After hybridization, based on the fluorescence intensity between amplicons obtained from “healthy” and “asthmatic” mice, an expression ratio (“asthmai”:“healthy”) was determined (table 1).

### Example 5

### By Way of Example One Novel Therapeutic Target Protein for the Treatment of Immune and/or Inflammatory Responses

The mRNA expression of gob 5 has been examined by PCR using gene-specific primer pairs (sense primer: GCCTTCGGACAGCAITACA; anti-sense primer TGCGTTGTCCAGGTGATAAG; product length 435 base-pairs). Gob 5 mRNA is present in lymph nodes, lung tissue, bronchoalveolar lavage cells and bone-marrow obtained from healthy BAL3/c mice. In tissues obtained from “asthmatic” mice compared to tissues obtained from “healthy” mice, the expression of gob 5 mRNA is increased in lymph nodes (approximately 4 fold), bronchoalveolar lavage cells (>10 fold), and bone marrow cells (approximately 2 fold). Mucus secreting goblet cells have never been described in lymph nodes or bone-marrow. The expression of gob 5 in murine bone-marrow derived mast cells and murine mast-cell lines is demonstrated (P815 and CFYTL-12). Additionally, a strong up-regulation of gob 5 in the dorsal root ganglia (DRG) obtained from the mouse asthma model was observed (FIG. 2). The expression of other members of the calcium activated chloride channel family was determined by PCR (table 1, Table 2 and FIG. 2). We have identified a murine homologue of CaCC3 (EST AA726662) and we show that the expression is strongly upregulated (>16 fold) in DRG of the mouse asthma model compared to healthy mice (FIG. 2). In contrast, the expression of the murine homologue (m_CaCC or m_CLCA1) of human CLCA3 was strongly down-regulated in DRG from the mouse asthma model (FIG. 2).

### Example 6

### By Way of Example One Novel Therapeutic Target Protein for the Treatment of Immune and/or Inflammatory Responses

LR8/CLAST1 belongings to the family of the tetraspanin (4TM) superfamily and has been discovered in a subpopulation of human lung fibroblasts- LR8 mRNA was not detectable by PCR in human smooth muscle cells, endothelial cells or epithelial cells. A murine homologue of LR8 (Signature sequence R1-OS-B1-D3) showed gene (i.e mRNA) expression in lymph nodes from mice and a down-regulation in the mouse asthma model. Bio-informatics analysis of the LR8 protein confirmed the presumed 4TM structure of the protein and revealed a striking homology with the beta chain of the high affinity IgE receptor (FceRI) (FIG. 3).

### Example 7

### Expression of Genes in a Second Mouse Model of Allergic Asthma

In order to validate the differentially expressed genes, a second, independent mouse model of allergic asthma was used. In this model, Balb/c mice are sensitized by two intraperitoneal injections of ovalbumin (OVA, 10 μg in 2.25 mg Alum adjuvant on day 0 and 7. Subsequently, the mice are exposed to three challenges (day 21, 24, 27) by inhalation of OVA (10 mg/ml) aerosol during 20 minutes. Thii model is characterized by high serum levels of OVA-specific IgE, strong airway eosinophilia, airway hyperresponsiveness to methacholine and goblet cell hyperplasia, concomitant with the appearance of Th2-like cells in lung tissue. Control sensitized mice are challenged by inhalation of saline and do not develop airway manifestations of asthma as described above. OVA sensitized Balb/c mice were challenged by inhalation of either saline or OVA aerosol and at 24 hours after the last challenge, we have isolated the lung, trachea, lung draining (thoracic) lymph nodes (TLN) and dorsal root ganglia (DRG) from these mice. Tissues were immediately stored in RNAlater (Ambion) and within one month transferred to Trizol (GibcoBRL) and total RNA was isolated according to the manufacturer's instructions. ds-cDNA was generated using the SMART-PCR cDNA synthesis kit (Clontech). DNA concentrations were determined spectrophotometrically. Subsequently, these cDNAs were serially two-fold diluted in the wells of 96-well microtiter plates, concentrations ranging from 1.5 ng/μl in sample 1, 0.75 ng/μl in sample 2, down to 0.73 pg/μl in sample 12 (2048×dilution of sample 1).

Five μl of each sample of each dilution series was used as input in a 20 μI PCR in the following buffer: 66.0 mM Tris-HCl (pH 8.8 at 25° C.); 4.0 mM MgCl2; 16.0 mM (NH4)2SO4; 33.2 μg/ml BSA; 340 μM of dGTP, DATP, dTTP and dCTP; and 0.02 Units/pd Taq polymerase (Gibco-BRL).

In table 7, a list of specfic primer pairs for the indicated genes is given. Two or three sets of primers were combined in each PCR-reaction: one of the two HPRT-primer-pairs and one or two gene-specific primer pairs. Each combination was chosen in such a way that fragments of clearly different lengths were obtained for each gene/EST or for the HPRT-oontrol. Also, primers were cross-checked in such a way that formation of primer-dimers was prevented (i.e., primer pairs with more than 4 bp of complementary sequences—especially when they were present at the end of a primer—were not used together in a PCR-reaction). Primer concentrations in the PCR-reactions were 0.5 μM for the gene/EST-specific primers. For the HPRT-primers, the concentrations used ranged from 0.3 μM down to 0.16 μM.

PCR was performed on a PCT100 (MJ research) or a PE9700 thermal cycler (Perkin Elmer), both with a heated lid (no oil used). A denaturation step of 3′ at 95 degrees Celsius was followed by 33-35 cycles of 30 sec 95 degrees Celsius, 40 seconds at 55 or 68 degrees Celsius (depending on primer sets used) and 2 minutes 72 degrees Celsius and then by a final 3 minutes at 72 degrees Celsius. After PCR, 5 pl loading dye was added to each sample and the whole samples were loaded onto 200 ml 2.5%/o Seakem LE-agarose-gels in 0.5×TBE in 50-well OwL electrophoresis trays and run at 80-100 Volt until the DNA's had migrated long enough to see each gene/EST-specific band (usually 1-2 hrs). Each gel was photographed with a CCD-camera. At least three photographs were taken from each gel at different diaphragm-settings. All pictures were stored electronically.

cDNA dilutions fom similar tissues obtained from differently treated mice (saline-vs. OVA-challenge) were loaded in such a way in the microtiter plates used to setup the PCR reactions that they would end up next to each other on the gel.

During the whole procedure described above, multichannel pipets were used to setup the PCR-reactions and to load the gels. Furthermore, master-mixes containing everything but the cDNAs (i.e., including the PCR-buffer, nucleotides, primers and Taq-polym erase) were prepared for each set of primer pairs used. In this way experimental variation is kept to a minimum. Also, one can be sure that the total absence of one specific band in one dilution-series is not an artifact if this band is present in another series setup with the same master-mix. To determine the level of differential expression, the patterns obtained on gel were scored by eye.

For the gene/EST specific bands and for the HPRT-control-band the highest dilution in which the band was still present was scored.

Using the RPRT-band as a reference the difference in gene-expression was scored as a “+1”, “+2”, “+3” etc., indicating that the gene/EST tested was over expressed at a respectively 2-fold, 4-fold, 8-fold etc. higher level in the OVA than in the saline-sample, or as “−1”, “−2”, “−3” etc., indicating that the gene/EST tested was over expressed at a respectively 2-fold, 4-fold, 8-fold etc. lower level in the OVA than in the saline-samples.

As an example in FIG. 1A, the results are shown for the genes with signature sequences OS-B1-C3 and OtS2-C5. The interpretation, based on careful visual inspection (if necessary using photo's taken at different diaphragm-settings) is given by the bars below the photograph: For both saline and OVA the HPRT-band is visible down to dilution number 12.

For OS-B1-C3 no band is visible in the saline-dilution-series, whereas the band can be seen down to dilution number 10 in the OVA-dilution series, indicating that the gene from which this EST is derived is expressed in the Dorsal Root Ganglia of OVA-challenged animals at least 1024-fold (2 to the power 10, in table 8, this is scored as a 10) more strongly than in saline-challenged mice.

For OtS2-C5 the band is visible in the first two dilutions ion the saline-series and in the first three dilutions in the OVA-series, indicating that the gene from which this EST is derived is 2-fold (2 to the power 1, in table 5, this is scored as a 1) higher expressed after OVA-challenge compared to saline-challenge.

In a similar manner in FIG. 1B the results for Cyp2f2 (signature sequence R1-OS-B1-A1) and Gob5 (signature sequence R1-SO-R1-C11) show that Cyp2f2 is highly, but not differentially expressed (scored as a 0 in table 5), whereas Gob5 is expressed after OVA-challenge at least 4096-fold stronger than after Saline-challenge (2 to the power 12, scored as a 12 in table 5).

### Example 8

### Expression of Genes in Prototypic Cell-Lines

Allergic asthma is a complex chronic inflammatory disease that involves the activation of many inflammatory and structural cells, all of which participate in the typical pathophysiological changes of asthma [Barnes, 1998 #6873]. Many inflammatory cells are recruited to asthmatic airways or are activated in situ. These include mast cells, macrophages, eosinophils, T lymphocytes, B lymphocytes, dendritic cells, basophils, neutrophils and platelets. It is now increasingly recognized that structural cells may also be important sources of mediators in asthma. Airway epithelial cells, smooth muscle cells, endothelial cells and fibroblasts are all capable of synthesizing and releasing inflammatory mediators Moreover, these cells may become major sources of inflammatory mediators in the airway and this may explain how asthmatic inflammation persists even in the absence of activating stimuli. We have analyzed the expression of many of the identified genes in relevant murine cell-types (table 6 ). A cell-line expressing the relevant gene and the encoding protein can be used for functional studies into the role of the gene/protein and can be used for the screening of a compound (agonist or antagonist) that modulates at least one of the functions of the gene/protein. Cell-lines were cultured according to guidelines from the “American Type Culture Collection” (www.atcc.org) or as described in literature. The primary dendritic cells were generated from bone-marrow cells cultured in the presence of interleuin-4 and granulocyte-macrophage colony-stimulating factor as described in literature [Masurier, 1999 #6874]. After culture, cells were harvested and total RNA was extracted using Trizol according to the manufacturer's instructions. 1 μg of total RNA was transcribed into cDNA in a volume of 20 μl. cDNA was used in PCR reactions using gene specific primer pairs (see table 4 ) with a denaturation step of 20′ at 95 degrees Celsius, followed by 35 cycli at 94 degrees Celsius for 20″; 55 degrees Celsius for 30″, and 72 degrees Celsius for 30″ and then by a final 2′ at 72 degrees Celsius. In some experiments, the cells were activated by a well-known stimulus for that cell-type (see table 6).

In table 6, the expression (+) or absence (−) of expression of a particular gene in the respective cell-line is shown.

The mouse calcium-activated chloride channels gob-5 and the murine homologue (EST AA726662) of human CLCA2 are expressed in a prototypic B-lymphoeyte cell-line (A20). This cell-line and other B-lymphocyte cell-lines or primary B-cell cultures can be used to determine one or more functions of these ion channels in these cells. Chloride channels are important for cell activation and adhesion. Blockade of one or both of the chloride channels can be used in B-lymphocyte mediated diseases such as auto-immunity, allograft transplant rejection, allergy and asthma (type I hypersensitivity) and type III hypersensitivity (Arthus reaction, Farmer's lung) in which the disease is at least partially dependent on antibody production such as auto-antibodies, antibodies to graft tissue or antibodies to allergens.

On the other hand, activation of one or both of these chloride channels can be used in infectious diseases or in combination with vaccines (to protect against infections (viruses, bacteria, fungi, or protozoa) to boost the protective B-lymphocyte mediated antibody response.

The mouse calcium-activated chloride channels gob-5 (human CLCA1 homologue) and the murine homologue (EST AA726662) of human CLCA2 and the murine homologue (EST W41083) of human CLCA4 are expressed in prototypic monocyte/macrophage cell-lines (J774A.1 and RAW264.7) either under baseline conditions (EST W4 1083) or upon activation. These cell-lines and other macrophage/monocyte cell-lines or primary macrophagelmonocyte cell cultures can be used to determine one or more functions of these ion channels in these cells. Chloride channels are involved in cell activation and adhesion. Macrophages/monocytes are important effector cells in both the innate and adaptive immune response. Macrophages/monocytes can take up antigens and present these after processing to T-lymphocytes. Macrophages/monocytes can also deliver co-stimulatory signals (B7 family members, CD40, cytokines) to lead to optimal T-cell activation. In particular the production of interleukin-12 by macrophages is important to direct T-lymphocyte responses into the type 1 direction. Type 1 T-lymphocytes are characterized by a particular set of cytokines including interferon-γ. Modulation of one or more of these chloride channels can be used to inhibit or stimulate particular monocyte/macrophage functions such as expression of co-stimulatory molecules (CD40, B7 members) or to inhibit or stimulate the production of cytokines such as interleukin-12 and -18. In this way, inhibition of macrophage function is beneficial in the treatment of Th1 mediated diseases such as auto-immunity and Crohn's disease. Vice versa, stimulation of macrophage function by modulation of these chloride channels is beneficial in the treatment of Th2 mediated diseases such as allergy, asthma, certain types of auto-immunity and ulcerative colitis or in the potentiation of vaccination strategies. Macrophages/monocytes are also an important source of inflammatory mediators such as oxygen radicals, nitric oxide and tumor-necrosis factor-α that play a role in immune responses. Modulation of chloride channels is effective in the liitation of the production and release of these mediators.

The selective expression of the gene with signature sequence SvO2-1-D10 in the prototypic mast-cell line (P815), the prototypic B-lymphocyte cell-line (A20) and the prototypic macrophage/monocyte cell-lines (J774A.1 and RAW264.7) demonstrates a potential role of this gene and the encoding protein in the cellular function of these cell types. Modulation of the expression or activity of this gene/protein is useful in diseases mediated by mast cells (allergy, asthma, multiple sclerosis etc.), mediated by B-lymphocytes (auto-immunity, allergy, asthma etc) or modulated by macrophageslmonocytes. These cell-lines or other cell-lines representing the same cell-type or primary cell-cultures can be used to determine gene/protein function and screening of a compound (agonist or antagonist) that modulates at least one of the functions of the gene/protein.

The selective expression of the gene with signature sequence OtS2-G2 in the mast cell-line CFTL12 and the primary dendritic cells as well as in the activated mast-cell line P815 and in the activated T-cefl line EL4 demonstrates a potential role of this gene and the encoding protein in the cellular function of these cell-types. Modulation of the expression or activity of this gene/protein is useful in diseases mediated by mast cells, T-lymphocytes or initiated by dendritic cells. Thes cell-lines or other cell-lines representing the same cell-type or primary cell-cultures can be used to determine gene/protein function and screening of a compound (agonist or antagonist) that modulates at least one of the functions of the gene/protein.

The selective expression of the gene with signature sequence R1-OS-B1-A3 in the prototypic mast cell-line P815 and in the activated prototypic B-lymphocyte cell-line A20 demonstrates a potential role of this gene and the encoding protein in the cellular function of these cell-types. Modulation of the expression or activity of this gene/protein is useful in diseases mediated by mast cells or B-lymphocytes. These cell-lines or other cell-lines representing the same cell-type or primary cell-cultures can be used to determine gene/protein function and screening of a compound (agonist or antagonist) that modulates at least one of the functions of the gene/protein.

The selective expression of the gene with signature sequence R1-OS-B1-A5 in the prototypic mast cell-line P815, the T-cell line EL4 and the prototypic macrophage/monocyte cell-line RAW264.7 demonstrates a potential role of this gene and the encoding protein in the cellular function of these cell-types. Modulation of the expression or activity of this gene/protein is useful in diseases mediated by mast cells, T-lymphocytes or macrophages/monocytes. These cell-lines or other cell-lines representing the same cell-type or primary cell-cultures can be used to determine gene/protein function and screening of a compound (agonist or antagonist) that modulates at least one of the functions of the gene/protein.

The selective expression of the gene with signature sequence OtS2-B9 in the in the T-cell line ELA, in the prototypic macrophage/monocyte cell-line J774A.1 and in primary dendritic cells demonstrates a potential role of this gene and the encoding protein in the cellular function of these cell-types. Modulation of the expression or activity of this gene/protein is useful in diseases mediated by T-lymphocytes or by macrophages/monocytes or initiated by dendritic cells. These cell-lines or other cell-lines representing the same cell-type or primary cell-cultures can be used to determine gene/protein function and screening of a compound (agonist or antagonist) that modulates at least one of the functions of the gene/protein.

The selective expression of the murine homologue (mCaCC, GenBank Acc. AF052746) of human CLCA3 in the prototypic lung type-II epithelial cell-line C10 demonstrates a potential role of this gene and the encoding protein in the cellular function of this cell-type. This cell-line or other cell-lines representing type-II epithelial cells such as the human A549 cell-line or primary cell cultures of this cell-type can be used to determine the gene/protein function and screening of a compound (agonist or antagonist) that modulates at least one of the functions of the gene/protein. Type II lung alveolar cells produce surfactant. A deficiency in alveolar surfactant causes respiratory distress syndrome (RDS). Modulation of the expression or activity of this gene/protein is useful in diseases mediated by type-II alveolar cells such as RDS.

The selective expression of murine DC-SIGN (signature sequence OtS1-B7) in the primary cultures of bone-marrow derived dendritic cells demonstrates a potential role of this gene and the encoding protein in the cellular function of dendritic cells. Bone-marrow derived dendritic cells or cell-lines representing dendritic cells such as XS52 cell-line or other primary cell cultures of this cell-type can be used to determine the gene/protein function and screening of a compound (agonist or antagonist) that modulates at least one of the functions of the gene/protein. Dendritic cells are so-called professional antigen-presenting cells (APC) and thus play a crucial role in the initiation and progression of immune- and inflammatory responses mediated by T-lymphocytes. Blockade of ??mDC-SIGN is beneficial in the treatment of T-lymphocyte mediated diseases such as allergy, asthma, COPD, auto-immune diseases, inflammatory bowel diseases, allograft rejection and infectious diseases.

### Example 9

### Identification of Full-Length Sequence of OtS1-B7

Steps in the Identification of the OtS1-B7 as the Murine Homologue of Human DC-SIGN

1. The identified cDNA fragment with signature sequence OtS1-B7 was used for BLAST analysis leading to two hits with mouse genomic sequences: GenBank acc. AC73804 and AC3706.

2. Gene prediction using GenScan

(http://bioweb.pasteur.fr/seqanal/interfaces/genscan.html), BLAST

(http:/twww.ncbi.nlm.nih.gov/BLAST/) and ClustalW

(http://www2.ebi.ac.uk/clustalw/) led to the construction of a 19619 bp long uninterrupted mouse genomic sequence, designated Contig1A. Contig1A consists of the following overlapping contigs present in AC073804 and AC73706:

nt 1-11054=nt 294022-305082 from AC073804

nt 11009-19619=nt 237022-228395 from AC073804 (reverse complement)

nt 1805-7790=nt 39946-34025 from AC073706 (reverse complement)

nt 6918-15759=nt 32026-23233 from AC073706 (reverse complement)

1. From contig1A, a gene comprising OtS1-B7, was derived. The characterization of this gene was based on in-silico bioinformatics analysis in combination with “wet” work in the laboratory as described below:

2. Gene-prediction combined with extensive BLAST-searches and multiple alignment analyses yielded a putative gene consisting of 10 exons and encoding an mRNA with a length of approximately 1200 bp (table 7 and FIG. 6).

3. Subsequently, primers were developed (Table 8) and used for PCR analysis of the OtS1-B7 gene from cDNA of thoracic lymph nodes obtained from OVA-challenged mice. All primer pairs used yielded fragments after PCR with the lengths predicted by the OtS1-B7-sequence. Sequencing of a set of these overlapping fragments, confirmed that the OtS1-B7 gene-sequence was predicted correctly: no differences with respect to the deduced sequence were found.

4. The OtS1-B7 gene comprises the OtS1-B7 fragment:

nt 8426-8463 identical to nt 1-38 of OtS1-B7 (3′-part of exon 7)

nt 8955-9106 identical to nt 39-190 of OtS1-B7 (exon 8)

nt 10386-10495 identical to nt 191-300 of OtS1-B7 (exon 9)

nt 11618-11732 identical to nt 301-415 of OtS1-B7 (5′-part of exon 10)

5. In order to obtain the 5′- and the 3′-end of the OtS1-B7 cDNA, a variant of the RACE (rapid amplification of cDNA-ends) was used. At the 5′-end the sequence was shown to have a 5′-UTR of 22 bp. Determination of the 3′-end revealed that apart from the predominant 1.2 kb transcript, an approximately 800 bp longer transcript was present. Both transcripts encode the same 325 bp ORF.

6. Based on (i) the strong homology (approximately 50%, see multiple sequence alignment, FIG. 10) of OtS1-B7-ORF with human DC-SIGN, and (ii) the selective expression of OtS1-B7 in the primary dendritic cells (see example 8) and (iii) the staining of spleen dendritic cells with antibodies to peptides derived from OtS1-B7-ORF (see example 70), we conclude that we have identified the muse homologue of human DC-SIGN, a Dendritic Cell specific ICAM-3 Grabbing Non-integrin.

7. The Genetic localization of OtS1-B7 was done by ePCR of the 196219 bp long Contig1A-sequence (http://www.ncbi.nlm.nih. gov/genome/sts/epcr.egi), which resulted in the identification of marker 440942 (GenBank acc. AI480608) This marker has been mapped to mouse chr 8 (WI-RH Map 13431.25 cR3000), in a chromosomal region syntenic with human 19p13.3, the chromosomal region where DC-SIGN has been located.

8. In the 16916 bp Contig1A contig one other gene was predicted by GenScan:

14290-14329 promotor

17688-17690 ATG-start codon

17688-18494 809 bp single exon, BLAST-searches with this exon show that it encodes a retrotransposon with approximately 3000 active copies in the mouse genome

18618- 18623 poly-Adenylation-site

9. Southern-hybridization of a number of restriction digests (see FIG. 7) of BALB/c genomic DNA was performed using a 1101 bp long Contig1A cDNA fragment comprising the whole coding region of OtB1-B7 plus 123 bp of the 3′-UTR. This probe was generated by PCR with primers 47 (nt 3655-3684 in Contig1A) and 51 (nt 11861-11891 in Contig1A) and 25 ng of it was radiolabeled with 50 μCi of 32P-labeled alpha-dATP using a Amersham multiprime labelingkit (RPN1600Z, AP Biotech) and then hybridized to alkali blotted BALB/c-restriction digests in Church hybridization buffer at 65 degrees Celsius for about 18 hrs, washed 2 times with 2×SSC/0.1% SDS at RT for a few minutes each, and 2 times in 2×SSC/0.1% SDS for 10 and 90′ espectively and autoradiographed at −70 degrees Celsius using intensifying creens for 2 and 5 days respectively.

10. Results and the interpretation of the Southern-hybridization are shown in FIG. 7 and 4. FIG. 7 shows the predicted restriction enzyme map for the OtS1-B7-gene. FIG. 8 shows the EtBr-staining of the restrction-digests used (panel A), the autoradiograph after 2 and 5 days exposure time (panels B and C) and the interpretation (panels D an E). Panel D shows a graphical representation of all the hybridizing bands, the thickness of the bands indicates their relative strengths as judged by eye using both exposures. Panel E shows the expected hybridization pattern based on the predicted restriction enzyme map (shown in FIG. 7). The thickness of the bands is drawn proportional to the length of the hybridizing region present in each restriction fragment.

11. We conclude that all bands derived from OtS1-B7 which are expected to hybridize with the probe used are indeed present, confirming the correctness of the structure of the predicted gene.

12. Also, for all 6 restriction enzymes used additional hybridizing bands can be observed. In all cases, these additional bands hybridize much more weakly. Therefore, we conclude that in addition to OtS1-B7 a second gene is present in the genome of BALB/c which shares homology to OtS1-B7. Because the lengths of the hybridizing bands for this second gene are different for all six enzymes used and because these bands hybridize much more weakly when compared to the hybridizing OtS1-B7-bands, we conclude that this second gene is distantly related or that it might be a pseudogene.

13. For OtS1-B7 itself we conclude that it is present as a single copy per haploid genome in the mouse.

### Example 10A

### Polyclonal Antibodies and Immunohistochemistry

Polyclonal antibdies were prepared to mDC-SIGN (signature sequence OtS1-B7) by immunizing rabbits with immunogenic peptides selected from the mDC-SIGN protein sequence. The peptides used for the immunizations were selected on the basis of extracellular localization and immunogenicity (Eurogentec, Belgium). KLH conjugated peptides used for antibody production:

AA 77.92+C: H2N-KTP NTF, RQK EQE KIL QC-CONH2 (17 AA) and

AA 275-289+C: H2N-SRF QKY WNR GEP NNI C-CONH2 (16 AA)

Peptides were synthesized and polyclonal antibodies were generated by Eurogentec according to their standard procedures. In short, peptides were synthesized by Fimoc chemistry and coupled to Keyhole limpet Hemocyanin (KLH). Both KLH coupled peptides were mixed and used to immunize (200 μg in Freund's adjuvant) two rabbits on day 0, 14, 28 and 56. Serum was obtained prior to immunization (pre-serum, day 0) and at 35, 66 and 87 days after immunization (immune serum).

Both rabbits generated antibodies to either of the peptides as demonstrated by an ELISA using the peptide as coat. The polyclonal antibodies were used for immunohistochemistry. Cryostat sections (5 μm) of trachea, thoracic lymph nodes, spleen and dorsal root ganglia were used for immunohistochemistry. After blocking by incubation with 10% normal goat serum, tissues were washed and incubated with different dilutions (1:1000 to 1:5000) of either pre-immune- or immune-serum (day 87). Thereafter, tissues were incubated with anti-rabbit immunoglobulin antibody (DAKO) and after washing tissues were incubated with substrate DAB (Sigma) according to the rnanufacturer's instructions. After fixation and counter staining with hematoxyline, tissues were analyzed by light microscopy.

In the spleen from naive mice, there is a strong and localized staining of marginal zone dendritic cells for mIDC-SIGN in the tissues incubated with immune serum compared to pre-immune serum (FIG. 8). Thus, there is expression of mDC-SIGN protein in spleen marginal zone dendritic cells. This strongly confirms that we have identified the murine homologue of human DC-SIGN.

In dorsal root ganglia from saline-challenged control mice, there is a very weak staining for mDC-SIGN in the tissues incubated with immune serum versus pre-immune serum (FIG. 9). In the dorsal root ganglia from OVA-challenged mice (as described in example 7), there is a very strong staining for mDC-SIGN in the tissues incubated with immune serum compared to pre-immune serum. Thus, there is a weak expression of mDC-SIGN protein in DRG from control mice and a very strong expression of mDC-SIGN protein in DRG from OVA-challenged mice. In the trachea from saline-chaUenged control mice, there is staining of epithelial cells for mDC-SIGN in the tissues incubated with immune serum versus pre-immune serum. In the trachea from OVA-challenged mice (as described in example 7), there is a strong staining of epithelial cells for mDC-SIGN in the tissues incubated with immune serum compared to pre-immune serum (FIG. 10). Thus, there is expression of mDC-SIGN protein in tracheal epitbelial cells from control mice and a stronger expression of MDC-SIGN protein in DRG from OVA-challenged mice.

In the thoracic lymph nodes CLLN) from saline-challenged control mice, there is staining of dendritic cells for mDC-SIGN in the tissues incubated with immune serum versus pre-immune serum (Figure II). In the TLN from OVA-challenged mice (as described in example 7), there is a strong staining of dendritic cells for mDC-SIGN in the tissues incubated with immune serum compared to pre-immune serum. Thus, there is expression of mDC-SIGN protein in TLN from control mice and a stronger expression of mDC-SIGN protein in TLN from OVA-challenged mice

Antibodies (mono- or polyclonal or fragments thereof) to DC-SIGN can be used for the isolation, staining (immunohistochemistry, flow cytometry) and functional studies using murine dendritic cells.

### Example 10B

### OtS1-B7 Blockage by Monoclonal Antibody FRTR9 Attenuates Allergen-Induced Airway Manifestations of Asthma

We examined whether treatment with ERTR9, a specific monoclonal antibody that binds to OtS1-B7 (Geitenbeek et al., 2002, Kang et al., 2003), was able to affect the induction of airway manifestations of asthma in a mouse model of allergic asthma. Balb/c mice were divided into two groups of 12 animals (group I and group II) including 6 treatment and 6 control animals. Treatment group I received an intraperitoneal injection of ERTR9 (2 mg) 1 h prior to sensitization and 1 h prior to the first of three OVA inhalation challenges. Treatment group II received an intraperitoneal injection of ERTR9 (1 mg) 1 h prior to the first of three OVA inhalation challenges only. Control group I received 2mg of rat IgM 1 h prior to sensitization and 1 h prior to the first of three OVA inhalation challenges, and control group II received lmg of rat IgM 1 h prior to the first of three OVA inhalation challenges only. The mice were sensitized with OVA in alum adjuvant 1 h after treatment with antibodies and again after 7 days, as described previously (Deurloo et al., 2001). Blood samples were obtained twenty days after treatment to determine OVA-specific serum IgE antibody levels. Subsequently, the airway responsiveness to the bronchospasmogenic stimulus methacholine was determined by whole body plethysmography as described earlier (de Bie et al., 2000). Starting on day 24, the mice were challenged three times, once every third day, by inhalation of OVA (2 mg/ml). Airway responsiveness to methacholine was determined 24 h after the last inhalation challenge, on day 31. The lungs were lavaged to determine the numbers of infiltrating inflammatory cells in the lung lumen and blood samples were obtained to determine OVA-specific serum IgE antibody levels.

Airway responsiveness to methacholine was significantly increased (P<0.05) after OVA challenge as compared to pre-challenge values in mice 10 treated with control antibody (FIGS. 18A and 18B). In group I, treatment with ERTR9 before sensitization and before challenge, attenuated the allergen-induced airway hyperresponsiveness to methacholine (FIG. 18A). In group II, mice treated with ERTR9 prior to challenge only, airway responsiveness to methacholine was also significantly increased after OVA challenge as compared to pre-challenge values (P<0.05). However, airway responsiveness in ERTR9-treated mice was significantly lower than in control mice (P<0.05) (FIG. 18B).

Serum levels of OVA specific IgE were increased after OVA challenge as compared to pre-challenge values (Table 9A and 9B). Interestingly, treatment with ERTR9 prior to sensitization and challenge inhibited the upregulation of serum OVA specific IgE levels after OVA challenge in group I (Table 9A). In group II serum IgE levels were increased in the treatment and control groups, however, serum IgE levels are reduced by almost 50% in the ERTR9-treated mice.

The number of infiltrating eosinophils in the BALF of mice treated with ERTR9 before sensitization and challenge (group 1) was significantly reduced compared to control mice (FIG. 19A). In mice treated with ERTR9 before challenge only (group II), the number of infiltrating eosinophils in the BALF was reduced by almost 50% (FIG. 19B).

It is concluded that the blockade of OtS1-B7 binding sites, by treatment with the monoclonal antibody ERTR9, attenuates allergen-induced airway manifestations of asthma. These data show that OtS1-B7 plays an important role in the initiation and progression of the OVA-induced immune- and inflammatory response.

### Example 11

### Generation of mDC SIGN “Knock-Out” Mouse

In order to study the role of mDC-SIGN (signature sequence OtS1-B7), a targeting construct was designed to knock-out this gene in mice. The targeting-construct contains a left arm encompassing part of intron 4, exon 5, intron 5 and part of intron 6, followed by a PGK-hyg cassette and a left arm, encompassing part of intron 8, exon 9, intron 9, exon 10 and several kb of the region downstream of OtS1-B7 (see FIG. 12). After electroporation of the construct into 129/OLA E14 ES-cells, hygromycin resistant clones will be screened by Southern analysis or LD-PCR to obtain clones which correctly targeted and which do not contain random insertions of the targeting construct (diagnostic restriction fragments and the hybridization probe to be used are indicated in the figure). Some of these clones will be used for blastocyst-injections after which they will be transferred to 129 mice to generate mDC-SIGN knock-out mice.

Further Examples of Use.

DC-SIGN can be blocked by mono- and polyclonal antibodies or fragments thereof directed against DC-SIGN (protein or peptide fragments); by the soluble protein ligands ICAM-2 en -3 or fragments thereof, by HIV gp120 or fragments thereof; by mannose carbohydrates such as mannan and D-mannose; fucose carbohydrates such as L-fucose; plant lectins such as concanavalin A; antibiotics such as pradimicin; sugars such as N-acetyl-D-glucosamine and galactose; and the MansGlcNAc₂ oligosaccharide of soybean agglutinin. Calcium-activated chloride channels (CLCA1-4) can be blocked by mono- and polyclonal antibodies or fragments thereof directed against the ion channel (protein or peptide fragments); known non-specific chloride channel antagonists such as 4,4′-diisothiocyanatostilbene-2,2′-disulphonic acid (DIDS), 4-acetamido-4′-isothiocyanostilbene-2,2′-disulfonic acid (SITS), 5-nitro-2-(3-phenylpropylamino)benzoic acid (NPPD), niflumic acid, and the anti-allergic drug cromolyn; Ion-channel toxins such as FTX-3.3 or synthetic analogues such as sFTX-3.3 and argiotoxin. Antibodies (mono- or polyclonal or fragments thereof) to murine DC-SIGN can be used for (i) staining of dendritic cells by immunohistochemistry, flow cytometry etc.; (ii) for isolating and/or purifying dendritic cells from a biological sample or a culture medium; (iii) functional studies into the role of DC-SIGN. A number of genes are strongly increased in expression in DRG's obtained from “asthmatic”, OVA challenged, mice compared to control, saline challenged, mice such as genes with signature sequence: SvO2-1-A11; Svo2-1-C8, R1-OS-B1-C3; OtS2-B9, R1-OS-B1-D6, SvO2-1-B7 (with KPI domain) and OtS1-B7. Blockade of one or more of these genes or the encoding proteins by selective antagonists inhibits the excitability of sensory neurons and thereby prevents or decreases (1) the neurogenic component of inflammatory diseases, (2) hyperalgesia during inflammatory responses and (3) cough due to airway inflammation. The calcium-activated chloride channels Gob-5 and the murine homologue (EST AA726662) of human CLCA2 as well as the gene OtS2-C3 (signature sequence ID) are upregulated in trachea derived from “asthmatic” mice compared to “healthy” control mice. Blockade of one or more of these genes or the encoding proteins is beneficial in the treatment of allergic airway diseases. The murine homologue (EST AA726662) of human CLCA2 (also called CaCC3) is strongly upregulated in DRG's obtained from “asthmatic” mice compared to “healthy” control mice This corroborates the data presented here in example 5. Dorsal root ganglia contain sensory nerve bodies that are involved in neurogenic inflammation which contributes to allergic inflammation and pain (inflammatory hyperalgesia). Interference with human calcium-activated chloride channel CLCA2 may limit neurogenic inflammation in asthma and other diseases with a neurogenic inflammatory component. Furthermore, cough, which is a prominent symptom of asthma, is believed to be a result of sensory nerve activation. Blockade of hCLCA2 (or the murine homologue) by selective antagonists inhibits the excitability of sensory neurons and thereby prevents or decreases (1) the neurogenic component of inflammatory responses, (2) hyperalgesia during inflammatory responses and (3) cough due to airway inflammation.

Role of DC-SIGN:

Immature dendritic cells (DCs) migrate from the blood into peripheral tissues where they capture and process antigens and subsequently migrate to lymphoid organs to either activate or tolerize T-lymphocytes in an antigen-specifc way. DCs play an important role in allergic sensitization as well as in the induction of antigen-induced airway manifestations of asthma. In agreement herewith, we recently showed that passive transfer of ovalbumin (OVA) primed spleen derived DCs strongly potentiates the development of allergic airway inflammation, airway hyperreactivity and Th2-associated cytokine production upon subsequent antigen inhalation. These data clearly demonstrate that DCs are key regulatory cells in the initiation and progression of Th2-dominated allergic airway responses Recently, a DC-specific receptor called DC-SIGN (DC-Specific ICAM-3 Grabbing Non-integrin) was identified. DC-SIGN is a mannose binding cell-surface receptor, member of the C-type lectin family and appears to be expressed exclusively by DCs. DC-SIGN mediates the interaction between DCs and resting T-cells via ICAM-3 and has recently been shown to be important in DC-induced proliferation of human resting T-cells in vitro. Moreover, DC-SIGN has also been shown to be involved in trans-endothelial migration of DCs via interaction with ICAM-2 on human vascular endothelial cells. These data suggest an important role for DC-SIGN in the trafficking of DCs. We have discovered the murine homologue of DC-SIGN by differential gene expression using lung-draining lymph nodes obtained from control and OVA-challenged mice. The full-length sequence of murine DC-SIGN shows strong (50%) homology to human DC-SIGN. Rabbit polyclonal antibodies to murine DC-SIGN derived peptides were generated and used for immunohistochemistry. The staining of spleen and lymphoid tissues from mice using this antibody demonstrate that murine DC-SIGN is expressed on DCs.

Another important function of DC-SIGN in the initial dissemination of HIV-1 shortly after infection. DC-SIGN is highly expressed on DC present in mucosal tissues and binds to the HIV-I envelope glycoprotein gp120. DC-SIGN does not enable viral entry into DC, but protects the virus until DC migrate to T cell-rich lymph nodes. Here, DC-SIGN promotes efficient infection in trans of CD4+T cells. Transmission of HIV-1 by DC to T cells is inhibited by the blocking antibodies against DC-SIGN.

Interestingly, we demonstrate the expression of DC-SIGN at the protein level using polyclonal antibodies in mouse airway epithelial cells and in sensory neurons present in dorsal root ganglia. Epithelial cells in the respiratory system are not passive bystanders during assault of the epithelial barrier but participate actively in the inflammatory response to defend the airway. Because epithelial cells are located at sites of contact with the external environment, they are often the first cells to interact with potential microbial pathogens. Indeed, bacterial adherence to epithelial cells may be a prerequisite for colonization and infection and through this interaction epithelial cells may have the opportunity to detect and respond to pathogens independent of signals from other cell types in the respiratory system. The capacity for epithelial cells to directly detect microbial pathogens and immediately initiate expression of genes directed toward defense may allow for more efficient activation of the inflammatory response. Although several molecules that participate in airway defense have been identified, the activation and coordination of factors that result in a rapid and effective inflammatory response at the epithelial surface are only beginning to be elucidated. One mechanism for epithelial cells to participate in airway defense is through coordination of leukocyte influx and activation by expression of adhesive surface proteins and secretion of chemotactic molecules (interleukin-8, eotaxin, RANTES). DC-SIGN can play an important role in both the adhesion of leukocytes expressing ICAM molecules (in particular ICAM-2 and ICAM-3) and in the adhesion of pathogens (bacteria, fungi, parasites and viruses). Antibodies to DC-SIGN or compound that block the interaction between either ICAM molecules and DC-SIGN or the interaction between sugar moieties or other surface molecules of pathogens and DC-SIGN can be used to prevent or treat infections with these pathogens. Blockade of DC-SIGN is useful as a treatment for allergic asthma, COPD or other inflammatory diseases of the airways.

Dorsal root ganglia contain sensory nerve bodies that are involved in neurogenic inflammation which contributes to allergic inflammation and pain (inflammatory hyperalgesia). Furthermore, there is strong and convincing evidence for interactions between the immune and peripheral nervous systems. Many regulatory molecules are candidate mediators for communication between inflammatory cells and nerves. There is substantial evidence that various immune (lymphocytes) and inflammatory cells (mast cells, eosinophils etc.) are in close contact with nerves. Lymphoid tissues, mucosal sites (gut, airway) and skin are densely innervated and contacts between nerves and inflammatory cells have been demonstrated. Mast cells are in close proximity to nerves in mucosa and skin and nerve stimulation has been reported to cause mast cell activation. Such data suggest a dynamic interplay between the immune and nervous systems during immune and inflammatory responses. In agreement herewith, receptors for various neurotransmitters, in particular neuropeptides (substance P, CGRP etc.) are present on all immune- and inflammatory cells. A close contact between these immune- and inflammatory cells and the neurons is required for these neuronal mediators to be effective. DC-SIGN is an important adhesion molecule on sensory nerves that can bind to ICAM molecules (ICAM-2 and -3) on immune and inflammatory cells thereby establishing the close contact required for this neuro-immune interaction. Blockade of DC-SIGN inhibits the neuronal component of immune- and inflammatory responses and is beneficial in inflammatory diseases such as autoimmunity, allergy, asthma, inflammatory bowel disease etc. Neurotropic viruses such as herpes simplex virus (HSV) and human immunodeficiency virus (HIV) can infect peripheral neurons. Cell-surface expression of DC-SIGN in neurons may be a an important step in the infection of neurons with neurotropic viruses such as HSV and HIV mediated by glycosylated viral envelope proteins. Compounds that inhibit the interaction between DC-SIGN and the viral glycoproteine are useful in prevention and treatment of these neurotropic viral infections.

The selective expression of CLCA2 in the prototypic human epithelial cell-line demonstrates a role of this gene and the encoding protein in the cellular function of these cell-types. Chloride channels play a role in production and secretion of mucus and chemotactic molecules (interleukin-S, eotaxin, RANTES) by epithelial cells. CLCA2 also can play a role in cellular adhesion. This cel-line and other epithelial cell-lines can be used to study the role of human CLCA2 gene or the encoding protein in lung epithelial cell function such as mucus production and secretion and can be used for the screening of compounds (agonist or antagonist) that modulates at least one of the functions of the gene/protein.

Blockade of this ion channel will inhibit mucus production and is therefore beneficial in the treatment of airway diseases associated with increased mucus production such as asthma and COPD. Compounds that upregulate the expression of CLCA2 in human epithelial cells are useful in the treatment of patients with cystic fibrosis which have a defect in cAMP-ediated chloride secretion.

A number of genes are strongly increased in expression in DRG's obtained from “asthmatic”, OVA challenged. mice compared to control, saline challenged, mice 10 such as genes with signature sequence: SvO2-1-A11; SvO2-1-C8, R1-OS-B1-C3; OtS2-B9, R1-OS-B1-D6, SvO2-1-B7 (with KPI domain) and OtS1-B7 Blockade of one or more of these genes or the encoding proteins by selective antagonists inhibits the excitability of sensory neurons and thereby prevents or decreases (1) the neurogenic component of inflammatory diseases, (2) hyperalgesia during inflammatory responses and (3) cough due to airway inflammation.

The calcium-activated chloride channels Gob-5 and the rnurine homologue (EST AA726662) of human CLCA2 as well as the gene OtS2-CS (signature sequence ID) are upregulated in trachea derived from “asthmatic” mice compared to “healthy” control mice. Blockade of one or more of these genes or the encoding proteins is beneficial in the treatment of allergic airway diseases.

The murine homologue (EST AA726662) of human CLCA2 (also called CaCC3) is strongly upregulated in DRG's obtained from “asthmatic” mice compared to “healthy” control mice. Dorsal root ganglia contain sensory nerve bodies that are involved in neurogenic inflammation which contributes to allergic inflammation and pain (inflammatory hyperalgesia). Interference with human calcium-activated chloride channel CLCA2 may limit neurogenic inflammation in asthma and other diseases with a neurogenic inflammatory component. Furthermore, cough, which is a prominent symptom of asthma, is believed to be a result of sensory nerve activation. Blockade of hCLCA2 (or the murine homologue) by selective antagonists inhibits the excitability of sensory neurons and thereby prevents or decreases (1) the neurogenic component of inflammatory responses, (2) hyperalgesia during inflammatory responses and (3) cough due to airway inflammation. It is demonstrated herein that the human lung epithelial cell-line expresses the CLCA2 gene constitutively (FIG. 13). The selective expression of CLCA2 in the prototypic human epithelial cell-line demonstrates a role of this gene and the encoding protein in the cellular function of these cell-types. Chloride channels play a role in production and secretion of mucus and chemotactic molecules (interleukin-8, eotaxin, RANTES) by epithelial cells. CLCA2 also can play a role in cellular adhesion. This cell-line and other epithelial cell-lines can be used to study the role of human CLCA2 gene or the encoding protein in lung epithelial cell function such as mucus production and secretion and can be used for the screening of compounds (agonist or antagonist) that modulates at least one of the functions of the gene/protein. Blockade of this ion channel will inhibit mucus production and is therefore beneficial in the treatment of airway diseases associated with increased mucus production such as asthma and COPD. Compounds that upregulate the expression of CLCA2 in human epithelial cells are useful in the treatment of patients with cystic fibrosis which have a defect in cAMP-mediated chloride secretion.

### REFERENCES

Bates, E. E., Fournier, N., Garcia, E., Valladeau, J., Durand, I., Pin, J. J., Zurawski, S. M., Patel, S., Abrams, J. S., Uebecque, S., Garrone, P. and Saeland, S. (1999). APCs express DCIT, a novel C-type lectin surface receptor containing an immunoreceptor tyrosine-based inhibitory motif. J. Immunol 163, 1973-83.

de Bie, J. J., Kneepkens, M., Kraneveld, A. D., Jonker, E. H., Henricks, P. A., Nijkamp, F. P., van Oosterhout, A. J. (2000). Absence of late airway response despite increased airway responsiveness and eosinophilia in a murine model of asthma. Exp Lung Res. 26(7):491-507.

Deurloo, D. T., van Esch, B. C., Hofstra, C. L., Nijkamp, F. P., van Oosterhout, A. J. M. (2001). CTLA4-IgG reverses asthma manifestations in a mild- but not in a more “severe” ongoing murine model. Am. J. Respir. Cell Mol. Biol. 25(6), 751-60.

Geitenbeek, T. B., Torensma, R., van Vliet, S. J., van Dtuijnhoven, G. C., Adema, G. J., van Kooyk, Y. and Figdor, C. G. (2000). Identification of DC-SIGN, a novel dendritic cell-specific ICAM-3 receptor that supports primary immune responses. Cell 100, 575-85.

Geitenbeek, T. B., Groot, P. C., Nolte, M. A., van Vliet, S. J., Gangaram-Panday, S. T., van Duijnhoven, G. C. F., Kraal, G., van Oosterhout, A. J. M., van Kooyk, Y. (2002). Marginal zone macrophages express a murine homologue of DC-SIGN that captures blood-borne antigens in vivo. Blood 100, 2908-16.

Kang, Y-S., Yamazaki, S., lyoda, T., Pack, M., Bruening, S. A., Kim, J. Y., Takahara, R., Inaba, K., Steinman, R. M. and Park, C. G. (2003). SIGN-R1, a novel C-type lectin expressed by marginal zone macrophages in spleen, mediates uptake of the polysaccharide dextran. Int. Immunol., 16, No. 2, 177-86.

Steinman, R. M. (2000). DC-SIGN: a guide to some mysteries of dendritic cells. Cell 100, 491-94.

TABLE 1
Identification of differentially expressed genes in “asthmatic”mice
compared with “healthy” control animals.

| | | | | |
|---|---|---|---|---|
| Signature | | | | |
| Sequence | Sequence/gene | Human homologue | Array¹ | Blot² |

a) Known genes up-regulated in “asthma” versus “healthy” mice.

| | | | | |
|---|---|---|---|---|
| R1-SO-R1- | Igγ | IgGγ | 2.09 | 10 |
| A11 | | | | |
| StO1-A10 | Igε | Igε | 2.08 | |
| SvO2-1-C11 | Igμ | Igμ | | |
| StO1-A12 | IgG1 H chain | IgG1 H chain | 2.20 | |
| R1-SO-R1-B7 | Igκ | Igκ | 2.36 | 4 |
| R1-SO-Ri-A7 | SLPI (secretory leukocyte | SLPI | 3.19 | |
| | protease inhibitor) | | | |
| R1-SO-R1-E7 | Tdt (terminal | Tdt | 3.65 | |
| | deoxynucleotidyl | | | |
| | transferase) | | | |
| StO1-B3 | CsA-19 | CsA-19 | 1.57 | |
| StO1-B5 | MHC-II (I^{A·d)} | MHC-II | 3.11 | |
| R1-SO-R1- | Gob-5 (ca²⁺ activated C1- | CaCC1/CLCA1 | 1.88 | 2 |
| C11 | channel) | | | |
| R1-SO-R1- | Pendulin | Rch1/Srp1α/ | 0.84 | 2 |
| E11 | | Importin-α | | |
| R1-SO-R1- | EST AA277412; | CDC42-GAP | 1.02 | 2 |
| A12 | AW910210; AI591665; | (GTPase-activating | | |
| | AA980800 | protein) | | |
| StO1-C1 | Aspartyl aminopeptidase | Aspartyl | 1.41 | |
| | | aminopeptidase | | |
| StO1-D3 | RA7O (mouse retinoic acid | SKAP-HOM | 0.77 | |
| | responsive gene) | (SKAP55 homologue) | | |
| SvO2-1-B7 | APLP2 (amyloid β | APLP2 | | |
| | precursor-like protein) | | | |
| SvO2-1-D8 | GDP-dissociation inhibitor | Ly-GDI | | |
| | (ly-GDI) | | | |
| SvO2-1-C4 | Plastin-2 (PLS2) | L-Plastin | | |
| SvO2-1-C12 | Ubiquitin/60s | | | |
| SvO2-1-A4 | H2-Oa (MHC-II) | HLA-DNA | | |
| SvO2-1-G3 | EST AI3274 12; AA140026 | RNA Polymerase-II | | |
| | | subunit (POLR2G) | | |
| SvO2-1-A8 | EST AW546508 | Phospholipase- | | |
| | | C γ2 (PLCγ2) | | |
| SvO2-1-D4 | EST AW044803; | Clathrin (CLTCL2) | | |
| | AA823969; AA869959 | | | |
| SvO2-1-D5 | EST BB000142 | Glutamyl-propyl-tRNA | | |
| | | synthethase (EPRS) | | |

b) Expressed sequenc tags (EST'S) up-regulated in “asthma” versus
“healthy” mice

| | | |
|---|---|---|
| SvO2-1-D10 | EST AI153476; | |
| | AA537538 | |
| SvO2-1-A11 | EST AI451488 | AW173082 |
| SvO2-1-C8 | EST AA023597; | |
| | AW476575 | |
| SvO2-1-E6 | EST AI587693; | |
| | AA499481; | |
| | AU080538 | |
| SvO2-1-f1 | EST C77954 | |

c) Known g nes down-r gulated in “asthsa” versus “healthy” mice

| | | | | |
|---|---|---|---|---|
| R1-OS-B1-B1 | PIN (protein inhibitor | Dynein | 1.44 | 0.7 |
| | of NnoS) | light chain | | |
| R1-OS-B1-A1 | CYP2F2 (cytochrome | CYP2F1 | 0.35 | 0.1 |
| | P450 naphthalene | | | |
| | hydroxylase) | | | |
| R1-OS-B1-B6 | IDH-α (NAD⁺ | NAD⁺ | 0.71 | 0.5 |
| | dependent isocitrate | dependent isocitrate | | |
| | dehydxogenase) | dehydrogenase | | |
| R1-OS-B1-G3 | Stat-1 | Stat-1 | 0.65 | 0.3 |
| R1-OS-B1-H1 | SEPP1 Selenoprotein P | SEPP1 | 0.52 | 0.5 |
| R1-OS-B1-C5 | Decorin | Decorin | 0.40 | 0.3 |
| OtS2-F2 | Cathepsin B | Cathepsin B | 0.56 | |
| OtS2-E6 | Gluthation-S- | Gluthation-S- | 0.40 | |
| | transferase mu 2 | transferase | | |
| | (Gstm2) | | | |
| OtS2-H2 | Breast heat shock 73 | HSP 70 | 0.60 | |
| | protein (Hsc7S) | | | |
| OtS2-B12 | Suiphated | Clusterin | 0.46 | |
| | glycoprotein-2-isoform | | | |
| | APOJ/Clu | | | |
| R1-OS-B1-D3 | LR8/CLAST1 | LR8 | 0.54 | 0.5 |
| R1-OS-B1-C1 | EST AW211263; | Mitochondrial | 0.55 | 0.7 |
| | AI194829; AI098607; | trifunctional protein | | |
| | W08910 | | | |
| R1-OS-B1-A2 | UBP43 (ubiquitin | ISG43 | 0.80 | 0.5 |
| | specific protein) | | | |
| R1-OS-B1-D5 | Ferritine | Ferritine | 0.45 | 1.0 |
| OtS2-B4 | Unidentified | | 0.50 | |
| | mitochondrial gene | | | |
| OtS2-A1 | Mitochondrial cyt-C | | 0.43 | |
| | oxidiase subunit I | | | |
| OtS2-C10 | Mitochondrial enoyl- | Mitochondrial | 0.34 | |
| | CoA hydratase (rat) | enoyl-CoA | | |
| | | hydratase | | |
| OtS2-A6 | AOP2 (antioxidant | AOP2 | 0.45 | |
| | protein 2) | | | |
| OtS2-D9 | IL-2R-γ | IL-2R-γ | 0.51 | |
| OtS2-A7 | EST AA475628 | TIS11d | 1.12 | |
| | | (early response gene) | | |
| | | /tristetraprolin | | |
| OtS2-C6 | HSP (84 kd heat shock | HSP 90 | 0.75 | |
| | protein) | | | |
| OtS2-A10 | IFNγR (interferon-γ | IFNγR | 0.32 | |
| | receptor) | | | |
| OtS2-C11 | Ornithine | Ornithine decarboxylase | 0.55 | |
| | decarboxylase (Odc) | | | |
| OtS1-C11 | Stearoyl-CoA | Stearoyl-CoA | 0.38 | |
| | desaturase 1 (SCD 1) | desaturase | | |
| OtS2-B10 | MUSLYSM4 (mouse | | 0.54 | |
| | lysozyme gene) | | | |
| OtS2-D8 | Calnexin | Calnexin | 0.61 | |
| R1-OS-B1-D6 | Plunc | Plunc | 0.39 | |

d) Expressed sequence tags (EST's) down-regulated in “asthma” versus
“healthy” mice.

| | | | | |
|---|---|---|---|---|
| OtS2-D3 | EST AI451901; AW826053; | | 0.74 | |
| | AA712022, partially similar | | | |
| | to mouse CR2 | | | |
| OtS2-D2 | EST AA423205, similar to | | 0.87 | |
| | X57528 mouse retinoic acid | | | |
| | receptor-alpha | | | |
| OtS2-D10 | Similar but not identical to | | 0.53 | |
| | mouse CD59 (complement | | | |
| | inhibitory protein) | | | |
| OtS1-B7 | EST AA543877; AA914211 | Similar but not | 0.43 | |
| | (similar but not identical to | identical to membrane | | |
| | macrophage lectin-2) | C-type lectin 2 | | |
| R1-OS-B1- | EST AA691014; AW321759 | | 0.84 | 0.5 |
| C3 | | | | |
| OtS2-G2 | Mouse JHL1 (AF165227) | | 0.58 | |
| R1-OS-B1- | EST AI450028, AW548213; | MUM2 (AF129332) | 0.83 | 0.25 |
| H6 | AA672579 | | | |
| R1-OS-B1- | EST AA512682; AI314236 | | 0.65 | 0.7 |
| AS | | | | |
| R1-OS-B1- | EST AA396183 (similar to | ROD1 | 0.66 | 0.5 |
| C4 | rat ROD1) | | | |
| R1-OS-B1- | EST AW490156 (similarity to | EST | 1.02 | 0.3 |
| A5 | dynein beta subunit) | AI358291; | | |
| | | AI623698 | | |
| R1-OS-B1- | EST AI835555 | | | 0.7 |
| B2 | | | | |
| OtS2-C1 | EST AA939676; AA125221; | | 0.77 | |
| | AA798681; AA869527 | | | |
| OtS2-D7 | EST AUO78971; AA178650; | | 1.60 | |
| | AA231343 | | | |
| OtS2-B9 | EST AA792488; AA177706 | | 0.37 | |
| OtS2-A9 | EST AA273304; AA270364; | AF143676 | 0.56 | |
| | AA671609 | (multi-spanning | | |
| | | nuclear envelope | | |
| | | membrane protein) | | |
| R1-OS-B1- | EST AI874718; AA498063; | | 0.81 | |
| C6 | AA615985 | | | |
| OtS2-C3 | EST AI788596; AI892968; | | 0.66 | |
| | AA939676 | | | |
| OtS2-B6 | EST AI1528153; AA982059; | | 0.67 | |
| | AW488424 | | | |
| OtS2-A12 | EST AA940560 (Rho-GAP | AF217507 | 0.65 | |
| | domain) | | | |
| OtS2-B3 | EST AL022972 | AW958031 | 1.43 | |
| OtS2-A5 | EST AA488598; AL118320; | | 1.08 | |
| | AI507121 | | | |
| OtS2-C4 | EST AW913417; AI647667 | | 0.68 | |

e) Genes down-regulated in “asthma” versus “healthy” mice.

| | | | |
|---|---|---|---|
| R1-OS-B1- | See fig. 4 for | 0.97 | 0.7 |
| E5 | sequence | | |
| OtS2-C5 | See fig. 4 for | 0.35 | |
| | sequence | | |

Array ¹: Expression ratio (asthma:healthy) obtained by hybridization of a cDNA micro-array with fluorescently labeled amplicons (Cy5 versus Cy3) derived from “asthma” and “healthy” mice.
Blot ²: Expression ratio (asthma:healthy) obtained by virtual northern blotting of amplicons and hybridization with fluorescently labeled specific, individual gene fragments.

TABLE 2
members of the calcium-activated chloride channel family.

| Human gene/protein | Murine homologue | Signature sequence |
|---|---|---|
| CaCC1/CLCA1 | Gob-5 | R1-SO-R1-C11 |
| CaCC2 | EST W41083 | |
| CaCC3/CaCL2 | EST AA726662 | |
| CLCA3 | CaCC/CLCA1 | |

TABLE 3
An example of some of the differentially expressed genes involved in
the regulation/activation of T-lymphocytes from table 1.

| Up-regulated | Signature | Down-regulated | Signature |
|---|---|---|---|
| genes/proteins | sequence | genes/proteins | sequence |
| CaA-19 | St-O1-B3 | IL2-R-gamma | OTS2-D9 |
| Pendulin | R1-SO-R1-E11 | IFN-γ-R | OTS2-A10 |
| RA70 | StO1-D3 | Stat-1 | R1-OS-B1-G3 |
| Ly-GDI | SV02-1-D8 | | |
| Plastin-2 | SV02-1-C4 | | |
| EST:RNA | SVO2-1-G3 | | |
| Polymerase-II | | | |
| subunit | | | |
| EST: Clathrin | SVO2-1-O4 | | |
| EST: Cdc42-GAP | R1-SO-R1-A12 | | |

TABL 4

Primer pairs used for semi-quantitative PCR
analysis of indicated (signature sequence)
genes and th ir respective product length.

| | sense | anti-sense | |
|---|---|---|---|
| Signature | primer | primer | product length |
| sequence | “forward” | “reverse” | (base pairs) |
| OtS1-B7 | ATGAGTGACTC | AAGAACAGGAA | 415 |
| | CACAGAAGCCA | GCAGAGCAGCT | |
| | AGATGCAG | GCAGGAC | |
| SvO2-1-B7 | 1: | 1: | 568 |
| | ATACACAGGCT | ATGATGAAGCC | 504 with KPI |
| | GTTCCCGTT | TCCCGTG | |
| | 2: | 2: | 336 without |
| | AAGTGGTGGAA | AAATGCTGGAT | KPI |
| | GACCGTGAC | GAGGGTCTG | |
| SvO2-1-D8 | TGGACCTTACT | ACTCTTCTGGT | 401 |
| | GGGGATCTC | GGGTGAGGA | |
| | ??? | | |
| OtS2-A6 | ATGCCCGGAGG | TGCCTGTCAGC | 514 |
| | TTTGCTTCT | TGGAGAGAG | |
| R1-OS-B1-D3 | AGTCAAAGTGG | CAAGAGCACAG | 197 |
| | CCTCCACAC | CTCACAAGC | |
| R1-OS-B1-A1 | CAGCCATCTTG | ACAGAGCGGCT | 508 |
| | CTTCTCCTC | CAGGATAAA | |
| R1-SO-R1-C11 | GCCTTCGGACA | TGCGTTGTCCA | 412 |
| | GCATTTACA | GGTGATAAG | |
| EST AA726662 | GGTTGAGCAGC | ATTGCCCACGG | 362 |
| | GAATGGAAGAG | CGCTATCCA | |
| | C | | |
| EST W41083 | AGCTAGTCCTT | TGTTGGATGGT | 654 |
| | CTGGACAACGG | CCCGAACTCAA | |
| | TGC | A | |
| mCaCC | ATTAGTCACAT | TGGGAGACGCT | 414 |
| | TTGACAGCGCT | GCCACTTGTAG | |
| | GCC | AT | |
| SvO2-1-D10 | TTTGAACCTCG | GCACCCATACT | 806 |
| | CCCACTGTG | GATAGCTCTCA | |
| SvO2-1-A11 | TCTTCCTTTGC | TTCCCCCCTCT | 418 |
| | TCAGACACACA | TTACTCCTGG | |
| | GG | | |
| SvO2-1-C8 | GAAGACGCCAC | TGAGAGTGGAG | 635 |
| | TGTTCCGAA | GCTGCCGTC | |
| SvO2-1-E6 | TCGACCCGAAT | TTTTCCCGCTT | 633 |
| | CTGTTTGCA | CCTGTCTCAG | |
| OtS2-D3 | TCAGAAGAAGC | ATCCTGCGGCA | 264 |
| | TTTGAACTTTG | GCAAAAA | |
| | G | | |
| OtS2-D10 | GAAGGTGTCTG | TGCATTCCGGC | 307 |
| | TGAAGCCTGTG | TACAGCATAGA | |
| | G | | |
| mCD59 | CAGTCACTGGC | TGCATTCCGGC | 250 |
| | GATCTGAAAAG | TACAGCATAGA | 370 (5′ UTR |
| | | | variant) |
| R1-OS-B1-C3 | ACAAGGCTTTA | GAGAGCCGGGA | 665 |
| | AGACTGCGACA | GAGTTTGCTAT | |
| | GC | | |
| OtS2-G2 | AGTGCACTTGC | ACAAGGGGGAG | 428 |
| | ATGGAGCTCA | AAGCAGCTG | |
| R1-OS-B1-H6 | GAGCTGACCAA | GCGGGCACAGA | 227 |
| | CATGGGTGC | GGATTCTTC | |
| R1-OS-B1-A3 | GATCAACGCAA | CTTTGCCCAAA | 210 |
| | GCTCTTGGC | ATAGAGCCA | |
| R1-OS-B1-C4 | ACACTGTTGGG | GACTGAAGCAG | 121 |
| | GAAAACGAG | CTCAAGACC | |
| R1-OS-B1-A5 | ACCGAGACCAA | GGCGAGGCTCC | 413 |
| | GCTGCAGTG | CACTTACTC | |
| R1-OS-B1-B2 | CTGAGGGGAGC | CCCAGTGGATG | 271 |
| | CTGCTGGAA | CCTGAAACA | |
| OtS2-C1 | CCTAAGCCCTC | TGATCCTCATT | 379 |
| | GGATTTTAC | GCAGAAGTTTA | |
| | | GCT | |
| OtS2-D7 | TTTTTCATGGC | CACCCCTCTGC | 403 |
| | TTCCTGCGG | GACAAGACA | |
| OtS2-B9 | GACCTGGACGA | AGAAAATTCAG | 150 |
| | GACCCTGGT | CCACTGCCA | |
| OtS2-A9 | TCAGGAACTGA | CTGGCTCTTCT | 280 |
| | GTTCTCCAG | CTTTACCCT | |
| R1-OS-B1-C6 | CATCAGAGCCA | GGAAGCATACT | 433 |
| | GCTATGCCG | TCTTGGCCTCA | |
| OtS2-C8 | GCGCTGGGATT | CCTTCCTGAAA | 442 |
| | TTACGTGTG | ACATGCCTAGG | |
| OtS2-B6 | TTTAAAAGGGA | TGGTGAAGGGT | 347 |
| | GGGGTGGCA | CTCTAGGGCA | |
| OtS2-A12 | GCATCTGTCGC | GCAAAACGTCT | 353 |
| | TTGGAAGGA | CCCTCCACC | |
| OtS2-B3 | AATGGGACTTC | GGCCGATTCCT | 375 |
| | ATGGCCTCC | TTGCAGAAA | |
| OtS2-A5 | AGCCCTGGACT | GCCTGGGCTGG | 298 |
| | GCAAAGCTC | GTAACAAGA | |
| OtS2-C4 | TGTTTACAGAC | CATCAAGTCTG | 307 |
| | TTTGCAACC | GTCTCTGAG | |
| R1-OS-B1-E5 | TTCTTTGTTAC | TTGCTGGCTTC | 250 |
| | CTCAGGGGC | TGTGACATC | |
| OtS2-C5 | GTGTTTAGCAT | AGATAACACCC | 237 |
| | CTGAGCCTC | CTGTGTGAG | |
| SvO2-1-F1 | AGTGGGGGACA | GGCTGGCTCTG | 853 |
| | TGAGGGTTGGC | GCTCTGCTTTT | |
| R1-OS-B1-D6 | GCAAGCTGATT | GGCTGCTGCGC | 383 |
| | TTCAGGCTGCC | ATTTTGGAAAA | |
| R1-SO-R1-A12 | ATTCAGTGCTT | TGGTTGGGTGC | 233 |
| | GCCGCAT | ACGATGT | |
| R1-OS-B1-B1 | GGTGATCAAAA | GAACAGAAGAA | 241 |
| | ATGCAGACATG | TGGCCACCT | |
| HPRT | GTTGGATACAG | GATTCAACTTG | 158 or |
| | GCCAGACTTTG | CGCTCATCTTA | 516 |
| | TTG or | GGC or | |
| | AGTCCCAGCGT | TGGCCTGTATC | |
| | CGTGATTAGCG | CAACACTTCGA | |
| | ATGA | GAGGT | |

1: primers used for PCR reactions of cDNA obtained from cell-lines (see table 6).
2: primers used for cDNA obtained from mouse tissues (see table 5) designed to detect APLP2 gene without or with the Kunitz protease inhibitor (KPI) domain.

TABLE 5
Difference in expression of the indicated gene in lung tissue, trachea,
thoracic lymph nodes (TLN) and dorsal-root ganglia (DRG) of OVA
sensitized mice challenged with OVA versus saline. The value indicated
in the table represents the difference in the number of two-fold dilution
steps. A value of “3” means that the expression in OVA challenged
mice is at least 2³ (=8) times higher than in saline challenged mice.
A value of “−3” means that the expression in OVA challenged mice
is at least 8 times lower than in saline challenged mice. See example 1
for a detailed explanation.

| Signature | | | | |
|---|---|---|---|---|
| sequence | Trachea | Lung | DUG | TLN |
| OtS1-B7 | 0 | 0 | 13 | 0 |
| SvO2-1-B7 | | | | |
| +KPI | 0 | −2 | 3 | 3 |
| −KPI | 0 | −2 | −2 | 3 |
| SvO2-1-D8 | 0 | 0 | 0 | 2 |
| OtS2-A6 | ND | ND | 0 | −1 |
| R1-OS-B1-D3 | 0 | 0 | −1 | 0 |
| R1-OS-b1-A1 | 2 | 0 | 2 | 0 |
| R1-SO-R1-C11 | 12 | 12 | ND | 0 |
| EST | 4 | 0 | 5 | −2 |
| AA726662 | | | | |
| EST W41083 | ND | ND | ND | ND |
| SvO2-1-D10 | 0 | 0 | 0 | 0 |
| SvO2-1-A11 | 0 | 2 | 3 | 3 |
| SvO2-1-C8 | 1 | 0 | 4 | 1 |
| SvO2-1-E6 | 0 | 1 | 1 | 1 |
| OtS2-D3 | −1 | 3 | 0 | 0 |
| OtS2-D10 | −1 | 3 | 0 | 0 |
| R1-OS-B1-C3 | 0 | −1 | 10 | 0 |
| OtS2-G2 | ND | ND | 0 | −1 |
| R1-OS-B1-H6 | 0 | 1 | −2 | 2 |
| R1-OS-B1-A3 | −1 | 1 | −1 | −3 |
| R1-OS-B1-C4 | 0 | 0 | 2 | 0 |
| R1-OS-B1-A5 | 0 | 2 | −3 | −3 |
| R1-OS-B1-B20 | 0 | 0 | −2 | 1 |
| OtS2-C1 | 0 | 0 | −2 | 2 |
| OtS2-D7 | 0 | 2 | 0 | 2 |
| OtS2-B9 | ND | ND | 3 | 0 |
| OtS2-A9 | 1 | 3 | −1 | 1 |
| R1-OS-B1-C6 | 0 | 1 | 1 | 1 |
| OtS2-C3 | 3 | 1 | −1 | −1 |
| OtS2-B6 | 0 | 1 | 0 | 0 |
| OtS2-A12 | 0 | 3 | 0 | 0 |
| OtS2-B3 | −1 | −1 | 1 | 0 |
| OtS2-A5 | 2 | −1 | −1 | −1 |
| OtS2-C4 | 0 | −1 | −1 | −2 |
| R1-OS-B1-E5 | −1 | 0 | −2 | 2 |
| OtS2-C5 | 0 | −2 | 1 | 0 |
| SvO2-1-F1 | 1 | −1 | 0 | 1 |
| R1-OS-B1-D6 | | | | |
| 383 bp | 0 | 0 | 4 | 0 |
| 310 bp | absent | absent | −5 | 1 |

ND: Not determined

TABLE 6
Expression of the specified gene in the indicated murine cell-line. “+”
indicates that the gene is expressed in the cell-line; “−”
indicates absence of expression of the specified gene in the cell-line.

| Signature sequence | P815 | CFTL12 | EL4 | 3D054.8 | DO11.10 | A20 | J774A.1 | RAW264.7 | C10 | 3T3 | DC |
|---|---|---|---|---|---|---|---|---|---|---|---|
| OtS1-B7 | − | + | − | − | − | − | − | − | − | − | + |
| SvO2-1-B7 | + | + | + | + | + | + | + | + | + | + | + |
| SvO2-1-D8 | + | + | + | + | + | + | + | + | + | + | + |
| OtS2-A6 | + | + | + | + | + | + | + | + | + | + | + |
| R1-OS-B1-D3 | + | + | + | + | + | + | + | + | + | + | + |
| R1-OS-B1-A1 | − | − | − | − | − | − | − | − | − | − | − |
| R1-SO-R1-C11 | − | − | − | − | − | + | +³ | +³ | − | − | − |
| EST AA726662 | − | − | − | − | − | + | +³ | − | − | + | − |
| EST W41083 | − | + | − | − | − | − | + | + | − | +¹ | − |
| mCaCC | ND | ND | ND | − | − | − | − | − | + | − | ND |
| SvO2-1-D10 | +¹ | − | − | − | − | + | + | + | − | − | − |
| SvO2-1-A11 | + | + | + | + | + | + | + | + | + | + | + |
| SvO2-1-C8 | + | + | + | + | + | + | + | + | + | + | + |
| SvO2-1-E6 | + | + | + | + | + | + | + | + | + | + | + |
| OtS2-D3 | + | + | + | + | + | + | + | + | − | + | − |
| OtS2-D10 | − | ND | − | − | + | + | − | + | + | + | ND |
| mCD59 | + | + | − | + | + | + | − | + | + | + | + |
| 5′UTR variant | − | − | − | − | − | + | − | + | − | − | − |
| R1-OS-B1-C3 | + | + | + | + | + | + | + | + | + | − | +¹ |
| OtS2-G2 | +¹ | + | +¹ | − | − | − | − | − | − | − | + |
| R1-OS-B1-H6 | + | + | + | + | + | + | + | + | + | + | + |
| R1-OS-B1-A3 | + | − | − | − | − | +² | − | − | − | − | − |
| R1-OS-B1-C4 | + | + | + | + | + | + | + | + | + | + | + |
| R1-OS-B1-A5 | + | − | + | − | − | − | − | + | − | − | − |
| R1-OS-B1-B2 | + | + | + | + | + | + | + | + | + | + | + |
| OtS2-C1 | + | + | + | + | + | + | + | + | + | + | + |
| OtS2-D7 | + | + | + | + | + | + | + | + | + | + | + |
| OtS2-B9 | − | − | + | − | − | − | + | − | − | − | + |
| OtS2-A9 | + | + | + | + | + | + | + | + | + | + | + |
| R1-OS-B1-C6 | + | + | + | + | + | + | + | + | + | + | + |
| OtS2-C3 | + | + | + | + | + | + | + | + | + | + | + |
| OtS2-B6 | + | + | + | + | + | + | + | + | + | + | + |
| OtS2-A12 | + | + | + | + | + | + | + | + | + | − | + |
| OtS2-B3 | + | + | + | + | + | + | + | + | + | + | + |
| OtS2-A5 | +¹ | + | + | + | + | + | + | + | + | + | − |
| OtS2-C4 | + | + | + | + | + | + | + | + | + | + | + |
| R1-OS-B1-E5 | + | + | − | + | + | + | +³ | + | + | + | + |
| OtS2-C5 | + | + | + | + | + | + | + | + | + | − | − |
| SvO2-1-F1 | + | + | + | + | + | + | + | + | + | + | + |
| R1-OS-B1-D6 | − | − | − | − | − | − | − | − | − | − | − |
| R1-SO-R1-A12 | ND | ND | ND | + | + | − | − | − | + | − | ND |
| R1-OS-B1-B1 | ND | ND | ND | + | + | + | + | + | + | + | ND |

ND: Not determined.
¹only after in vitro activation with PMA (10 ng/ml) for three hours.
²only after in vitro activation with an activating anti-CD40 monoclonal antibody (clone 3/23; 1 μg/ml) for three hours.
³only after in vitro activation with lipopolysaccharide (1 μg/ml) and interferon-γ (50 U/ml) for three hours.
Murine cell-lines used: P815: Mast cell
CFTL12: Mast cell
EL4: T-lymphocyte
3DO54.8: T-helper lymphocyte
DO11.10: T-helper lymphocyte
A20: B-lymphocyte
J774A.1: Macrophage, Monocyte
RAW264.7: Macrophage, Monocyte
C10: Lung type II epithelial cell
3T3: Fibroblast
DC: Primary bone-marrow derived dendritic cell

TABLE 7
Annotation of Contig1A (19619 bp) of the gene comprising the
OtS1-B7 fragment. Numbers refer to the nucleotide position in Contig 1A.

| | | |
|---|---|---|
| 3633 3654 | 5′-UTR | |
| 3655 3657 | ATG-startcodon | |
| 3655 3700 | 46 bp exon 1 | |
| 3701 3808 | | 108 bp intron 1 |
| 3809 3895 | 87 bp exon 2 | |
| 3896 4420 | | 525 bp intron 2 |
| 4421 4510 | 90 bp exon 3 | |
| 4511 5011 | | 501 bp intron 3 |
| 5012 5092 | 81 bp exon 4 | |
| 5093 6149 | | 1057 bp intron 4 |
| 6150 6236 | 87 bp exon 5 | |
| 6237 7064 | | 828 bp intron 5 |
| 7065 7151 | 87 bp exon 6 | |
| 7152 8376 | | 1225 bp intron 6 |
| 8377 8463 | 87 bp exon 7 | |
| 8464 8954 | | 491 bp intron 7 |
| 8955 9106 | 152 bp exon 8 | |
| 9107 10385 | | 1279 bp intron 8 |
| 10386 10495 | 110 bp exon 9 | |
| 10496 11617 | | 1122 bp intron 9 |
| 11618 11966 | 349 bp exon 10 | |
| 11618 12784 | 1167 bp exon 10A | |
| 11766 11768 | TAG-stopcodon | |
| 11769 11966 | 3′-UTR | |
| 11946 11950 | poly adenylationsite | |
| 11769 12784 | 3′UTR | |

| | |
|---|---|
| 12769 12773 | poly adenylationsite |
| | differential splicing yields 2 mRNA's of |
| | ca 1198 and 2016 bp, respectively, both encoding |
| | the same 325 bp ORF |

TABLE 8

Primers used for the characterization of the gene
comprising OtS1-B7. “F” in the primer name refers
to forward or sense primer; “R” in the primer
name refers to reveree or anti-sense primer.

| Position | | |
|---|---|---|
| in contig1A | primer name | sequence |
| 3642-3659 | 0055-FOTS1-B7F | GACAGCGGCAACCATGAG |
| 3647-3678 | 0054-FOTS1-B7F | CGGCAACCATGAGTGACTCCAC |
| | | AGAAG |
| 3655-3684 | 0047-OTS1-B7F | AAGAACAGGAAGGAGAGCAGCT |
| | | GCAGGAC |
| 4450-4478 | 0048-OtS1-B7-R | AGCTGGGTCAGTTCCTGGAGGA |
| | | TCTTCTCT |
| 5049-5078 | 0049-OtS1-B7-R | AGCTGGGTCAGTTCCTGGAGGA |
| | | TCTTCTCT |
| 5078-5092 + | 0050-OtS1-B7-R | GGGGATCCTGGACGTAAGCTCA |
| 6150-6163 | | TCTGTCA |
| 4506-4510 + | 0017-F-OtS1-B1 | CCAAGTCTCCAAAACCCCAAAT |
| 5012-5038 | | ACCGAGAGGC |
| 5012-5041 | 0032-OTS1-B7F | TCTCCAAAACCCCAAATACCGA |
| | | GAGGCAGA |
| 10393-10422 | 0033-OTS1-B7F | TGCAGCAGGCTTCTAAGGCTAA |
| | | AGGACCAA |
| 11636-11665 | 0034-OTS1-B7R | TCCTCACCGATGTTGTTAGGCT |
| | | CCCCTCTA |
| 11666-11695 | 0010-R-OtS1-B7 | CAGCCATCCCCAGCAAATTCGA |
| | | CACAGTCT |
| 11682-11711 | 0052-OtS1-B7-F | GCTGGGGATGGCTGGAATGACT |
| | | CTAAATGT |
| 11735-11763 | 0053-OtS1-B7-F | CAAGAAGTCTGCAACCCCATGC |
| | | ACTGAAG |
| 11769-11796 | 0056-OtS1-B7-R | ATGGCATGAAGGTAGGAGCGGA |
| | | GATGAG |
| 11781-11828 | 0057-OtS1-B7-R | CGAAAGTGAGGCACATCCAT |
| 11861-11891 | 0051-OtS1-B7-R | AAGAAGAATCCCAGAGCCTTTT |
| | | TCACGATCC |
| 11862-11881 | 0124-OtS1-B7-F | GATCGTGAAAAAGGCTCTGG |
| 12183-12202 | 0126-OtS1-B7-F | TGGCTAGATGTTCCCACCTC |
| 12832-12851 | 0125-OtS1-B7-R | TTCCTGCAGGGATGAGCTAC |
| 12558-12577 | 0127-OtS1-B7-R | TCAGATCACCAGCCTTGCTA |
| 12753-12779 | 0140-OtS1-B7-R | CAGGACTTTATTACAGCAACAG |
| | | TAAAC |

TABLE 7

Primer pairs used for PCR analysis of human
calcium-activated chloride channel family members
and HPRT in human H292 lung epithelial cells.

| | | | product |
|---|---|---|---|
| | | anti- | length |
| Gene | sense primer | sense primer | (base pairs) |
| CLCA1 | TGCAGACAGTTGAGC | CCCCAAAAGCATCAA | 417 |
| | TGGGGTCCT | TGAGGCC | |
| CLCA2 | AAATTCATACCTTCG | CTGGCCTGCCACGTA | 568 |
| | TGGGCATTGC | ACTAGAAACA | |
| CLCA4 | GCAAAACATTTCCTG | TGAGGCCATTGTTCT | 421 |
| | CTGCAGACTG | GAGCCTTCATC | |
| HPRT | TGCTGAGGATTTGGA | TGACCAAGGAAAGCA | 368 |
| | AAGGGTGTTT | AAGTCTGCAT | |

TABLE 8
members of the calcium-activated chloride channel family.

| Human gene/protein | Murine homologue |
|---|---|
| CLCA1/CaCC1 | Gob-5 |
| CLCA2/CaCC3/CaCL2 | EST AA726662 |
| CLCA3 | CaCC/CLCA1 |
| CLCA4/CaCC2 | EST W41083 |

TABLE 9A
Serum levels of OVA specific IgE (Units/ml). Balb/c mice (n = 5 or 6
per group) were treated with control antibody or ERTR9 antibody prior
to sensitization and prior to the first of three OVA inhalation challenges
(group I). Levels of OVA specific IgE were measured before and after
OVA inhalation challenge. Values are expressed as mean ± SEM.
Serum IgE levels after challenge are significantly increased *P<0.05
as determined by the student's t-test.

| Treatment | Control | ERTR9 |
|---|---|---|
| Before challenge | 606.4 ± 59.2 | 484.2 ± 62.1 |
| After challenge | 1739.6 ± 202.3* | 992.3 ± 301.7 |

TABLE 9B
Serum levels of OVA specific IgE (Units/ml). Balb/c mice (n = 5 or
6 per group) were treated with control antibody or ERTR9 antibody
prior to the first of three OVA inhalation challenges (group II).
Levels of OVA specific IgE were measured before and after OVA
inhalation challange. Values are expressed as mean ± SEM.
Serum IgE levels after challenge are significantly increased *P<0.05
as determined by the student's t-test.

| Treatment | Control | ERTR9 |
|---|---|---|
| Before challenge | 203.6 ± 40.62 | 190.0 ± 23.8 |
| After challenge | 17257.2 ± 4088.7* | 9684.7 ± 2596.0* |

### FIGURE LEGENDS

FIG. 1: Effects of by-GDI and Cdc42-GAP on small GTP-binding proteins Rac and Cdc42

FIG. 2: PCR products using cDNA obtained fom dorsal root ganglia (DRG) isolated from “healthy” or “asthmatic” mice. PCR was carried out using conditions well known in the art using the gene-specific primer pairs for

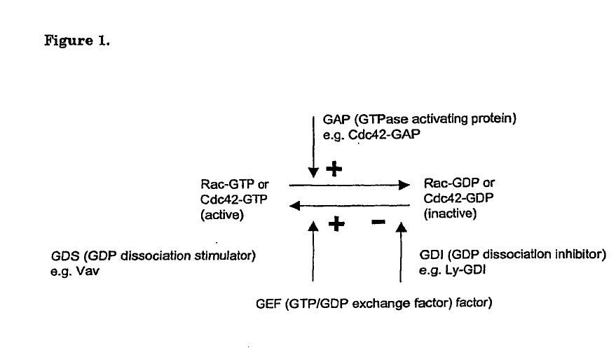

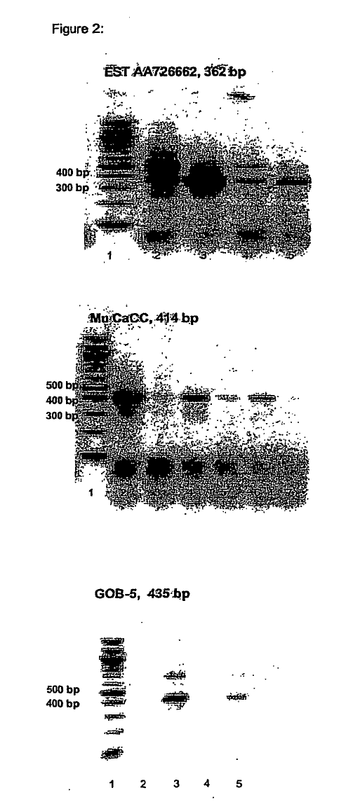

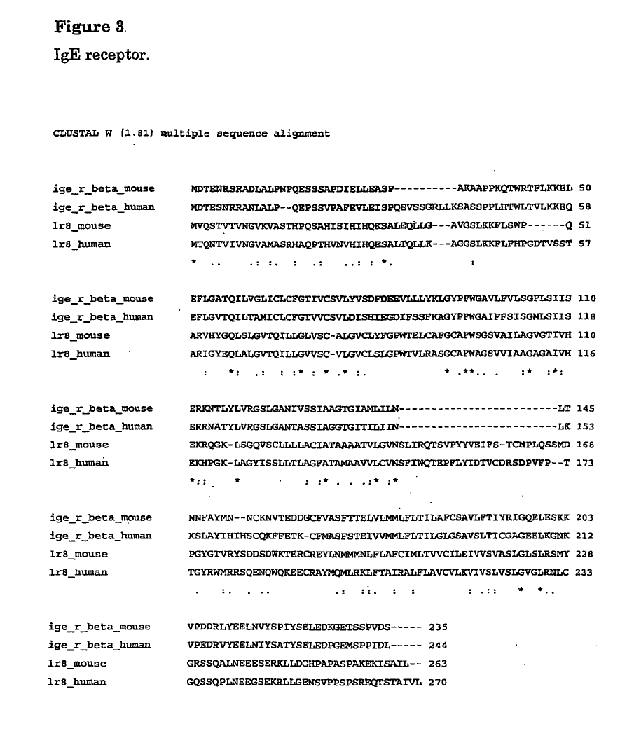

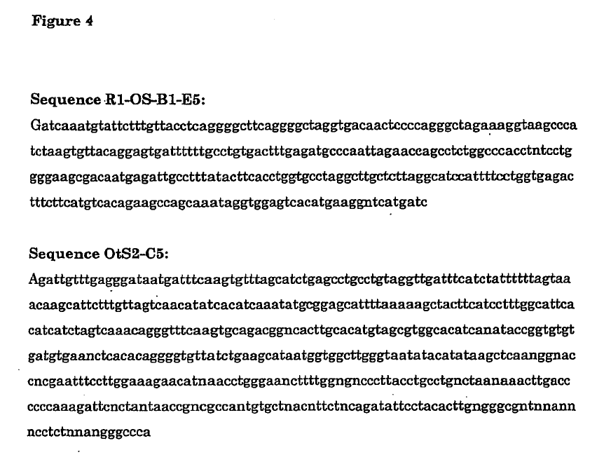

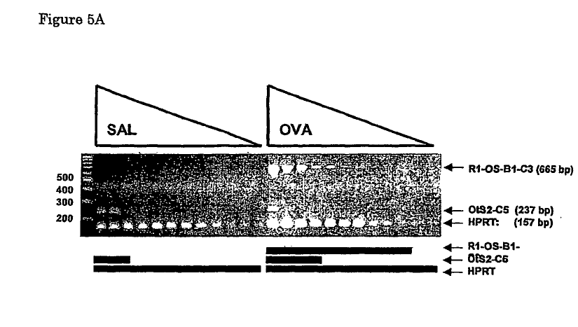

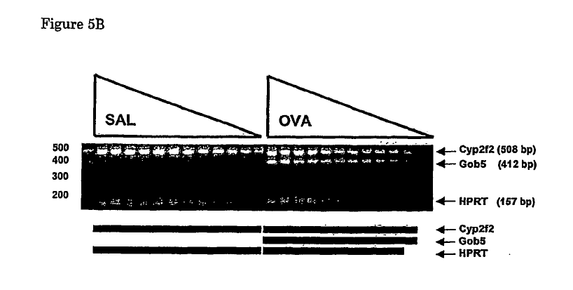

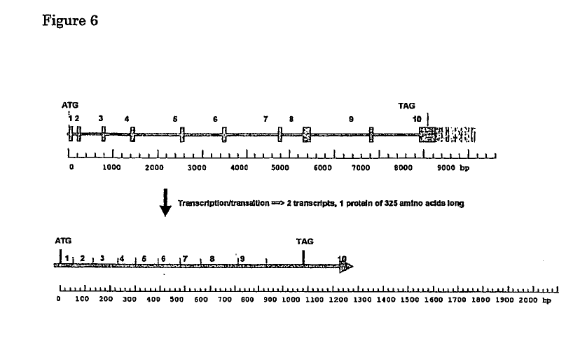

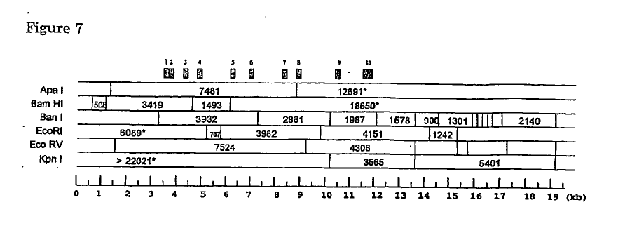

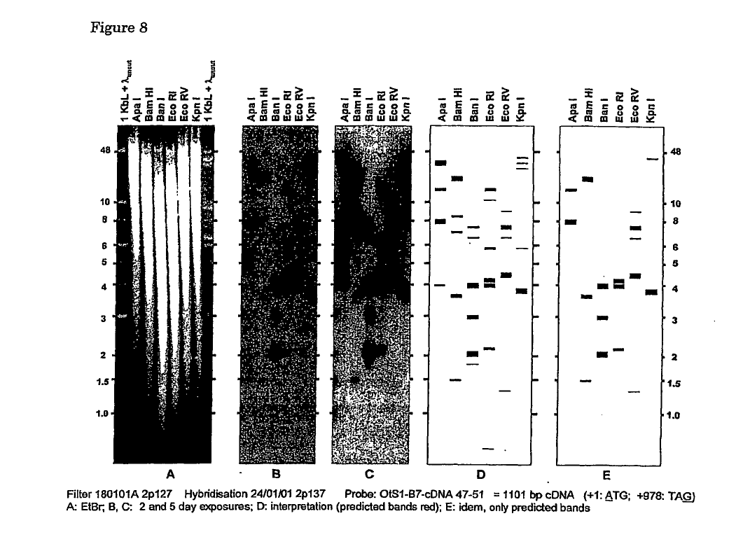

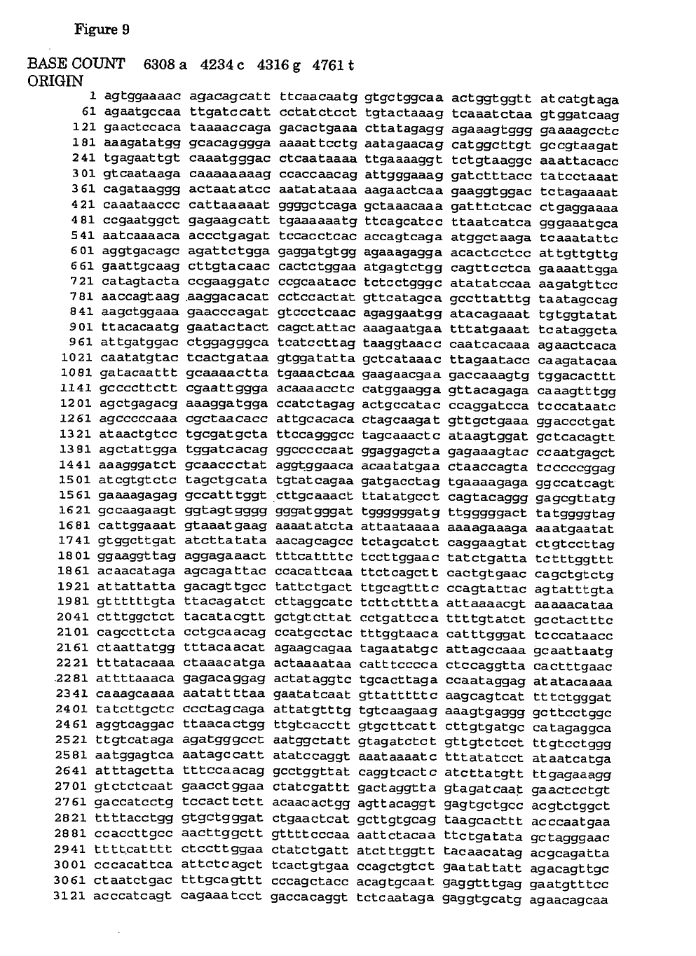

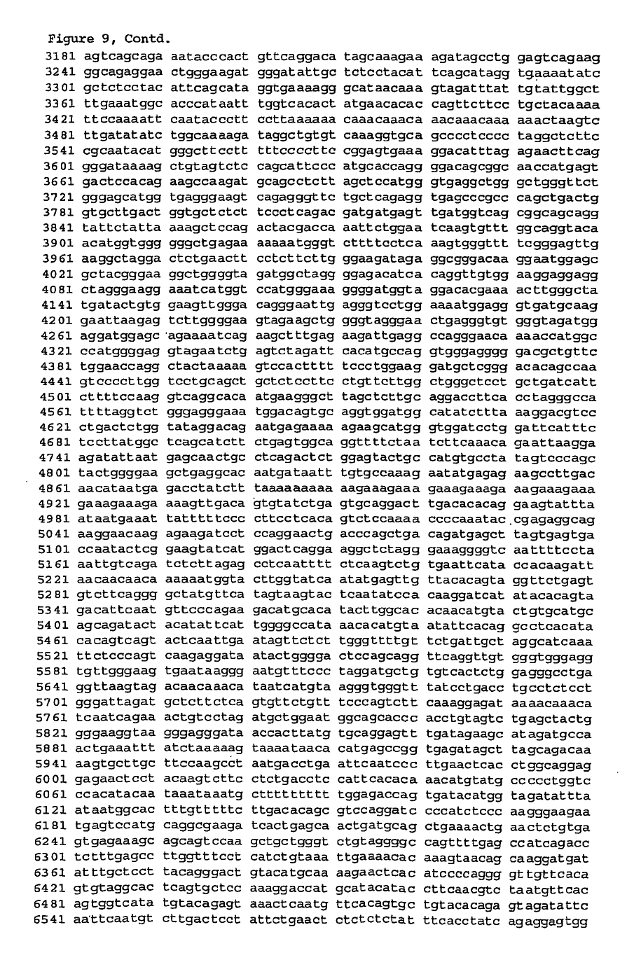

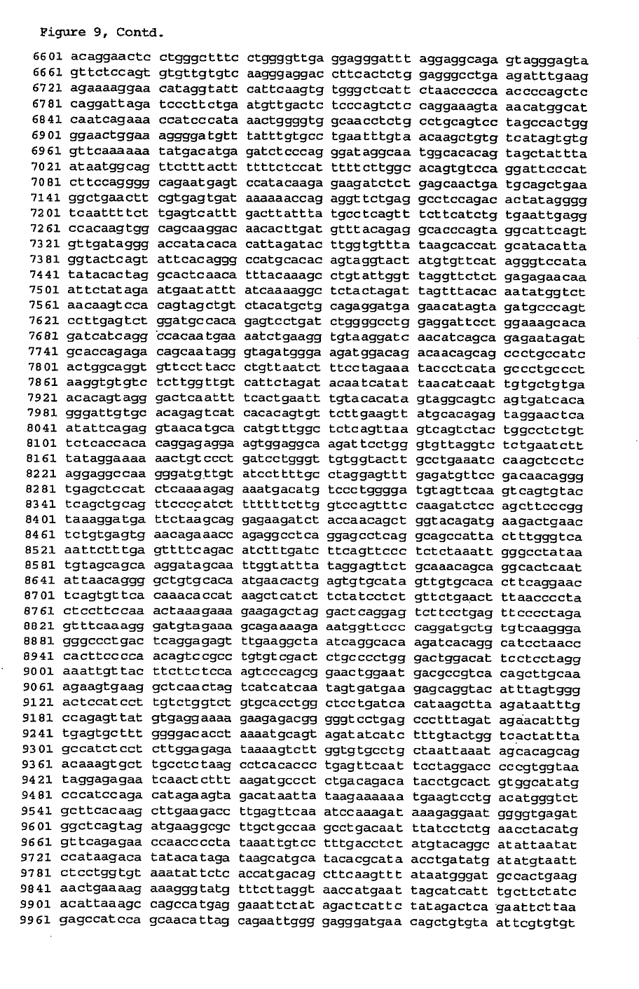

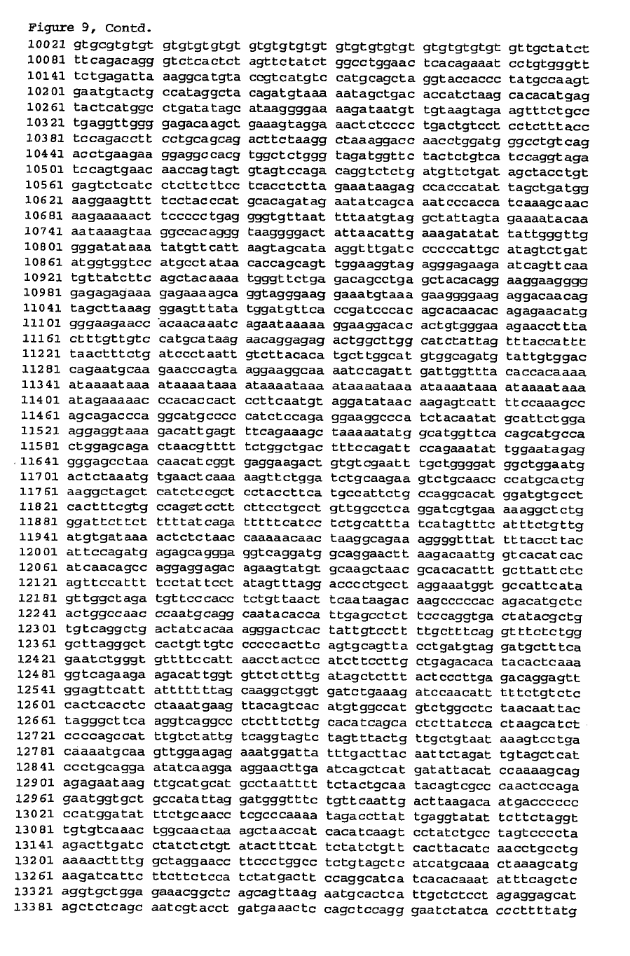

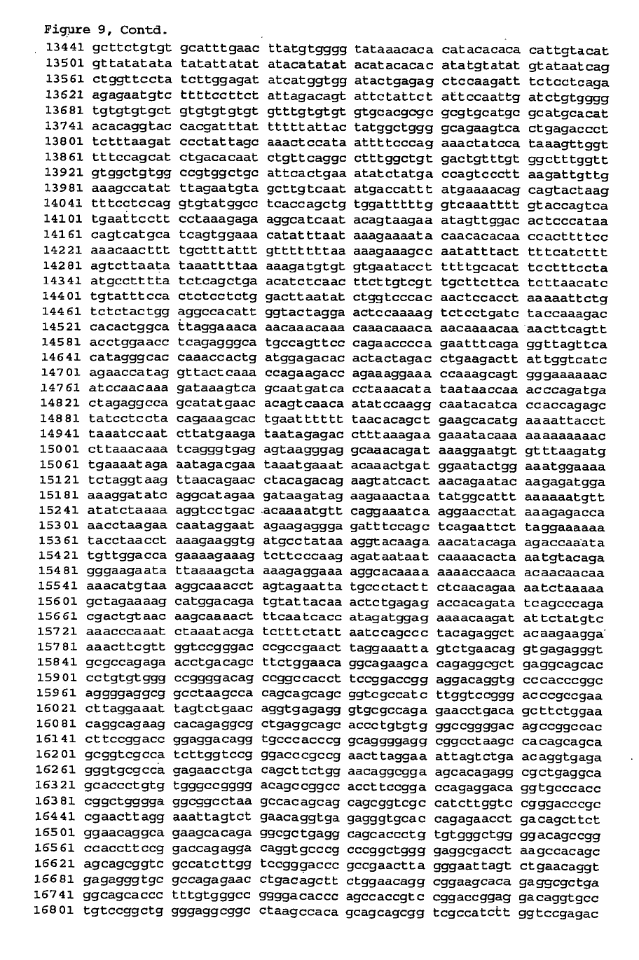

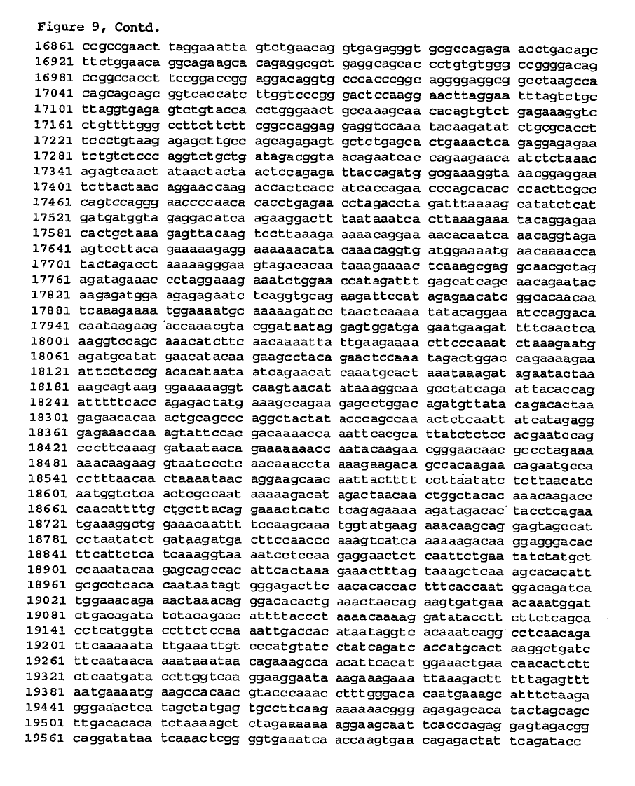

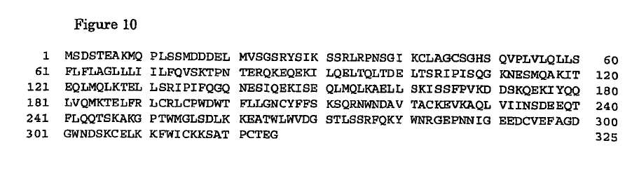

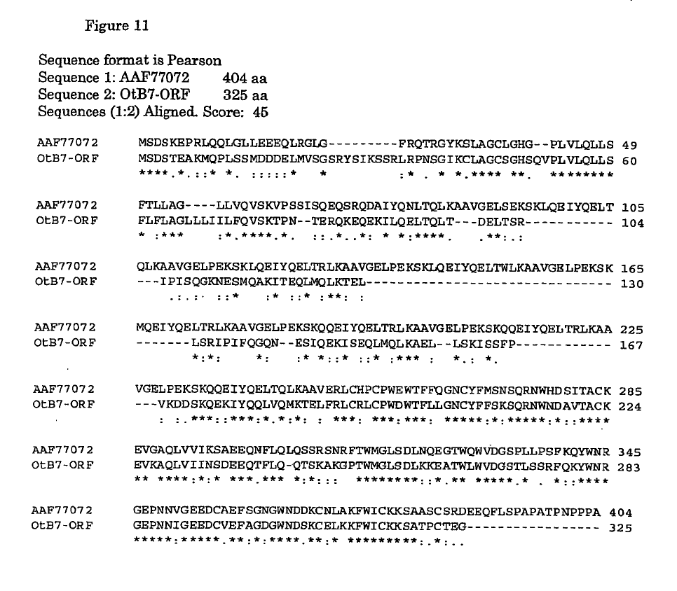

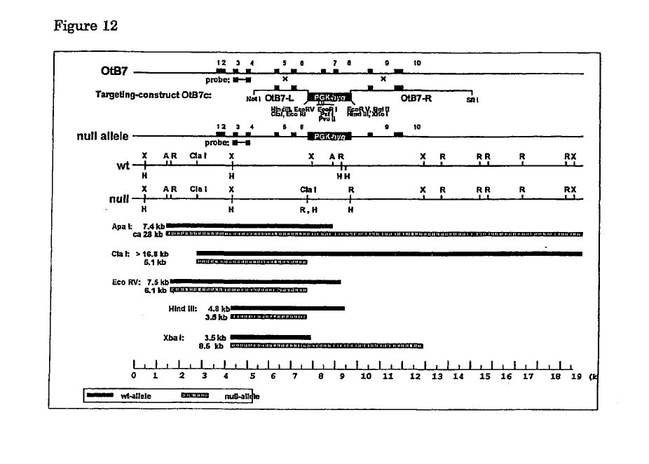

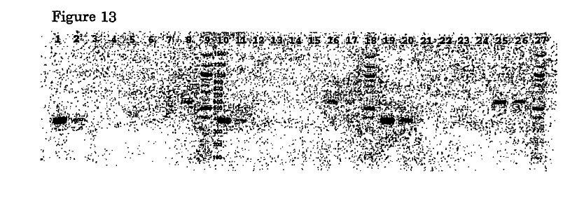

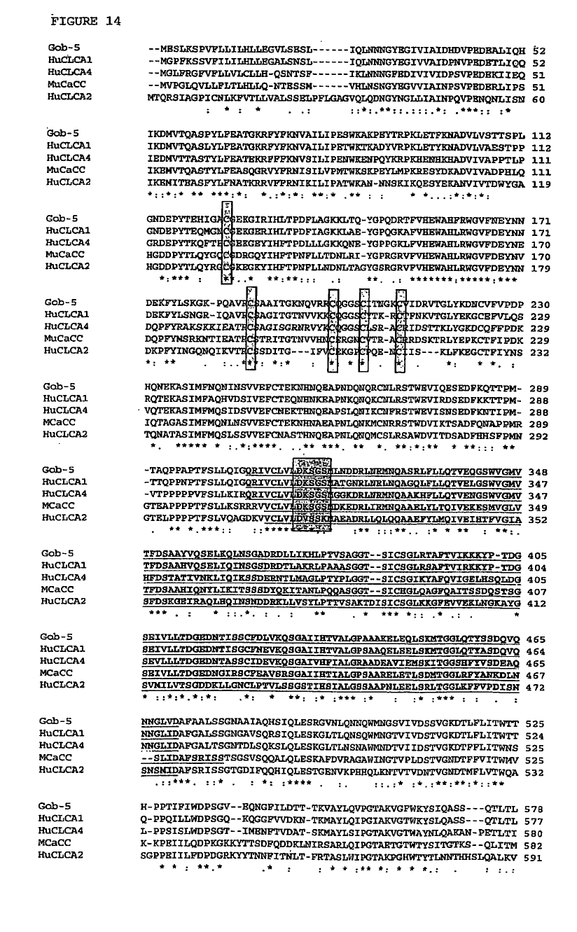

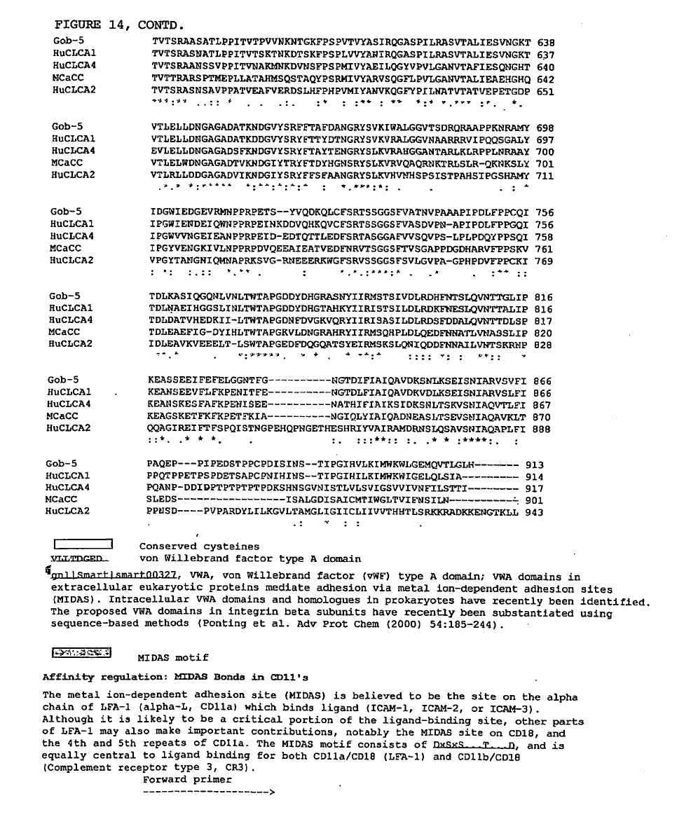

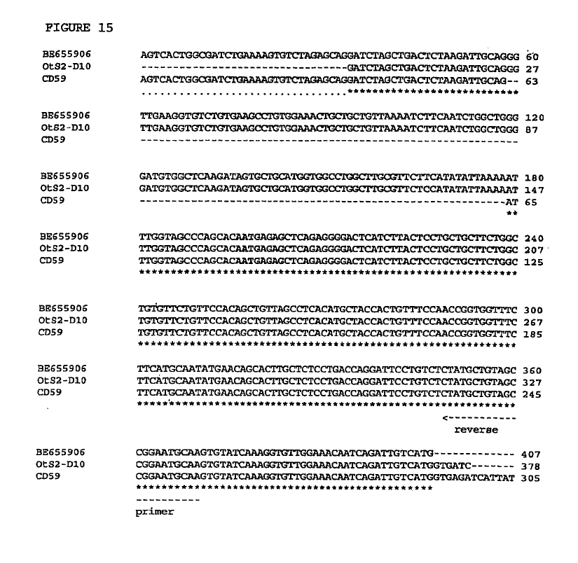

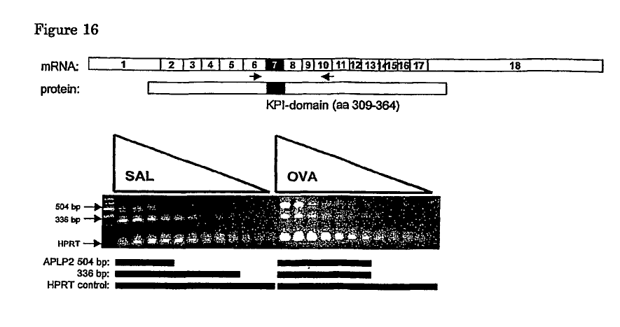

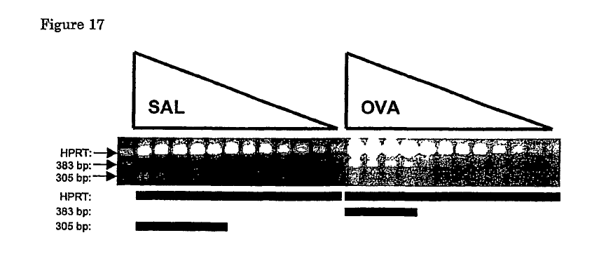

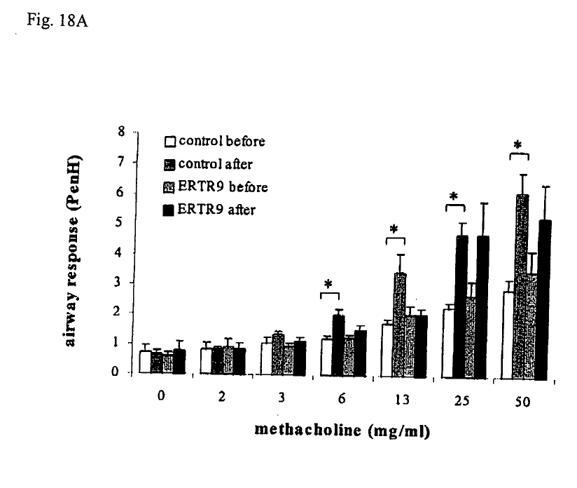

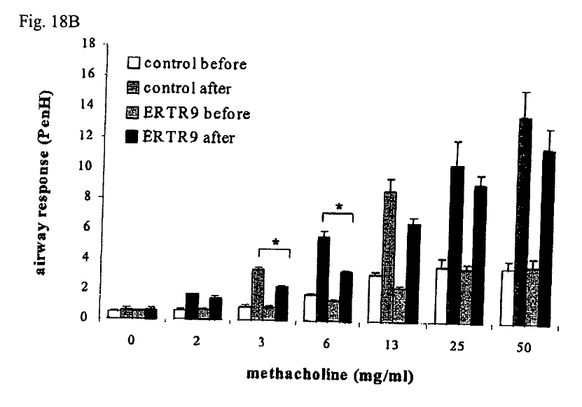

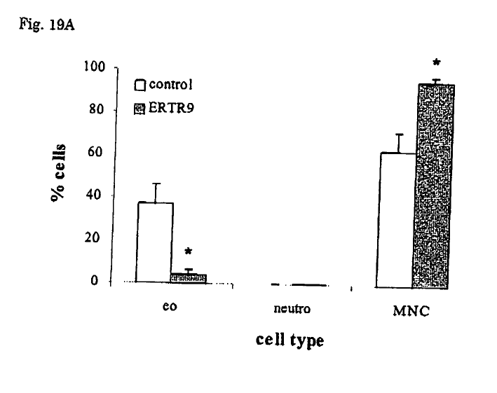

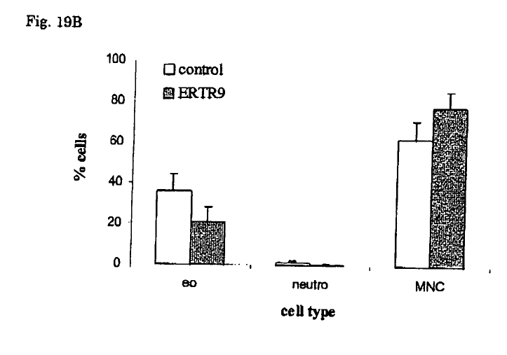

a) EST AA726662 (Top)(sense primer. GGTTGAGGAGCGGAATGGAAGAGC; antisense primer: ATTGCCCACGGCGCTATCCA, product length 362 base pairs);

b) m_CaCC (Middle)(sense primer: ATTAGTCACATTTGACAGCGCTGCC; antisense primer: TGGGAGACOCTGCCACTTGTAGAT, product length 414 base-pairs); and for

c) gob 5 (Bottom)(sense primer: GCCTTCGGACAGCATTTACA; anti-sense primer TGCGTTGTCCAGGTGATAAG; product length 435 base-pairs).

Lane 1 refers to 100 bp DNA ladder; lane 2, 4 and 6 refers to cDNA obtained from DRG of “healthy” mice and prediluted respectively ¼, 1/16 and 1/32; lane 3, 5 and 7 refers to cDNA obtained from DRG of “asthmatic” mice and prediluted respectively ¼, 1/16 and 1/32.

FIG. 3. Homology between LR8 and the beta chain of the high affinity IgE receptor.

FIG. 4. Genes down-regulated in “asthma” versus “healthy” mice.

### FIGURE LEGENDS

FIG. 5A: PCR analysis of gene-fragments with signature sequences R1-OS-B1-C3 and OtS2-C5 using cDNA from dorsal root ganglia obtained from saline- (SAL) or ovalbumin (OVA) challenged mice as described in example 1. HPRT house-keeping control gene is used to control for the relative amount of cDNA. Two-fold dilution series from left to right. The black bars indicate the dilutions that gave a PCR product.

FIG. 5B: PCR analysis of genes Cyp2f2 and Gob-5 using cDNA from lung tissue obtained from saline- (SAL) or ovalbumin (OVA) challenged mice as described in example 1. HPRT house-keeping control gene is used to control for the relative amount of cDNA. Two-fold dilution series from left to right. The black bars indicate the dilutions that gave a PCR product.

FIG. 6: Gene comprising OtS1-B7 fragment.

FIG. 7: Restrictionmap of Contig IA, the gene comprising OtSi-B7 fragment.

FIG. 8: EtBr-staining of the restriction-digests used (panel A), the autoradiograph after 2 and 5 days exposure time (panels B and C) and the interpretation (panels D an E). Panel D shows a graphical representation of all the hybridizing bands, the thickness of the bands indicates their relative strengths as judged by eye using both exposures. Panel E shows the expected hybridization pattern based on the predicted restriction enzme map (shown in FIG. 3). The thickness of the bands is drawn proportional to the length of the hybridizing region present in each restriction fragment.

FIG. 9 Complete sequence of Contig IA, the gene comprising OtS1-B7 fragment. Contig1A consists of the following overlapping contigs present in Genbank acc. AC073804 and AC73706:

nt 1-11054=nt 294022-305082 from AC073804

nt 11009-19619=nt 237022-228395 from AC073804 (reverse complement)

nt 1805-7790=nt 39946-34025 from AC073706 (reverse complement)

nt 6918-15759=nt 32026-23233 from AC073706 (reverse complement)

FIG. 10: The protein (325 AA) encoded by the predicted gene encompassing Contig1A comprising the OtS1-B7 fragment.

FIG. 11: CLUSTAL W (1.81) multiple sequence alignment of the polypeptide derived from the gene comprising OtS1-B7 (OtS1-B7-ORF) and DC-SIGN (Genfank acc.nr. AAF77072, also designated CD209.

FIG. 12: Outline for the generation of a genetically engineered null-mice for murine DC-SIGN (signature sequence OtS1-137, indicated as OtB7 in the figure), the mouse homologue of human DC-SIGN.

FIG. 13: Lanes 1-8 represent PCR products obtained using non-stimulated human lung epithelial cell-line H292. Lanes 10-17 represent PCR products obtained using PMA (10 ng/ml for 3 hours) stimulated H292 cells. Lanes 19-26 represent PCR products obtained using IL-9 (U/ml for 3 hours) stimulated H292 cells. PCR products in lanes 1,2,10,11,19,20 represent housekeeping enzyme HPRT. PCR products in lanes 3,4,12,13,21,22 represent Calcium activated Chloride Channel 1 (CLCA1). PCR products in lanes 5,6,14,15,23,24 represent CLCA4 PCR products in lanes 7,8,16,17,25,26 represent CLCA2 Lanes 9,18,27 represent a 100 bp ladder.

FIG. 14: ClustalW analysis of calcium-activated chloride channels. Indicated are conserved cysteines, the von Willebrand factor type A domain and the MIDAS motif. CLUSTAL W (1.81) multiple sequence alignment

FIG. 15: ClustalW analysis of CD59, signature sequence OtS2-D10 and EST (GenBank acc. BE655906. Indicated are the forward and reverse primers as described in Table 1 (example 1). CLUSTAL W (1.81) multiple sequence alignment

FIG. 16: Top: Schematic representation of APLP2 mRNA and protein with the KPI domain (exon 7) and the slected primer pair to identify splice variants with or without the KPI-domain. Bottom: PCR analysis of cDNA from dorsal root ganglia obtained from saline (SAL)- or ovalbumin (OVA) challenged mice as described in example 1). HPRT house-keeping control g ne is used to control for the relative amount of DNA. The black bars indicate the dilutions that gave a PCR product.

FIG. 17: PCR analysis of murine Plunc (signature sequence R1-OS-B1-D6) of cDNA from dorsal root ganglia obtained from saline (SAL)- or ovalbumin (OVA) challenged mice as described in example (1). HPRT house-keeping control gene is used to control for the relative amount of DNA. Two-fold dilution series from left to right. The black bars indicate the dilutions that gave a PCR product.

FIG. 18A: Airway responsiveness to aerosolized methacholine was measured in conscious, unrestrained mice (group I) 24 h after the third OVA inhalation challenge. Airway responsiveness in control mice (first and second bar) and ERTR9 treated mice (third and fourth bar) was measured before (plain bars) and after (striped bars) the OVA aerosol challenge period. Values are expressed as mean±SEM. Post challenge airway responsiveness is significantly increased in control mice. * P<0.05 as determined by the student's t-test.

FIG. 18B: Airway responsiveness to aerosolized methacholine was measured in conscious, unrestrained mice (group II) 24 h after the third OVA inhalation challenge. Airway responsiveness in control mice (first and second bar) and ERTR9 treated mice (third and fourth bar) was measured before (plain bars) and after (striped bars) the OVA aerosol challenge period. Values are expressed as mean±SEM. Post challenge airway responsiveness is significantly reduced in ERTR9-treated mice compared to controls. * p<0.05 as determined by the student's t-test.

FIG. 19A: Cellular composition of the BALF was determined 24 h after the third OVA inhalation challenge. Control mice (white bars) and ERTR9 treated mice (grey bars). Values are expressed as mean t SEM. *P<0.05 as determined by the student's t-test. Eo: eosinophils; neutro: neutrophils; MNC: mononuclear cells.

FIG. 19B: Cellular composition of the BALF was determined 24 h after the third OVA inhalation challenge. Control mice (white bars) and ERTR9 treated mice (grey bars). Values are expressed as mean±SEM. Eo: eosinophile; neutro: neutrophils; MNC: mononuclear cells.

What is claimed is:

1. A nucleic acid library comprising:
genes or a functional fragment thereof, said genes or functional fragment thereof essentially capable of, directly or indirectly, modulating an immune response observed with airway hyperresponsiveness and/or bronchoalveolar manifestations of asthma.

2. The nucleic acid library of claim 1 wherein the immune response is up-regulated.

3. The nucleic acid library of claim 1 wherein the immune response is down-regulated.

4. The nucleic acid library of claim 1, claim 2, or claim 3 wherein said nucleic acid library comprises a nucleic acid essentially equivalent to a signature sequence as shown in Table 1, Table 2 or Table 3.

5. The nucleic acid library of claim 1, claim 2, claim 3, or claim 4 wherein at least one of said genes encode a molecule selected from the group consisting of a regulatory molecule, a co-stimulatory molecule, an adhesion molecule, a receptor molecule, a calcium activated chloride channel, a DC-SIGN molecule involved in modulating an immune response, and combinations thereof.

6. A method for modulating an immune response in an individual, the method comprising:
modulating a gene comprising a nucleic acid at least functionally equivalent to a nucleic acid identifiable by a signature sequence as shown in Table 1, Table 2 or Table 3.

7. The method according to claim 6 wherein said gene modulates a signal transduction cascade pertaining to an immune response in the individual.

8. The method according to claim 7 wherein said signal transduction cascade modulates the production of cytokines, chemokines, growth factors, or combinations thereof.

9. The method according to claim 6, claim 7, or claim 8 wherein said gene modulates an action selected from the group consisting of sensory nerve activation, a Th1 mediated immune response, a Th2 mediated immune response, the generation of anti-oxidants, the generation of free radicals, a CDS⁺ T-lymphocyte response, or combinations of any thereof.

10. The method according to claim 6, claim 7, claim 8, or claim 9, wherein the gene encodes a gene product capable of modulating an immune response.

11. The method according to claim 6, claim 7, claim 8, claim 9, or claim 10, wherein said immune response includes airway hyperresponsiveness and/or bronchoalveolar manifestations of asthma.

12. The method according to claim 6, claim 7, claim 8, claim 9, claim 10, or claim 11, wherein the gene is modulated by transducing a cell of the individual.

13. A substance capable of modulating a gene, said substance comprising:
a nucleic acid at least fuinctionally equivalent to a nucleic acid identifiable by a signature sequence as shown in Table 1, Table 2 or Table 3.

14. A medicament comprising the substance of claim 13 in a pharmaceutically acceptable form and present in an amount sufficient to produce a therapeutic effect.

15. A method of treating an immune response observed with airway hyperresponsiveness and/or bronchoalveolar manifestations of asthma in a subject, the method comprising administering the substance of claim 14 to the subject.

16. A process for producing an antagonist against a proteinaceous substance encoded by a nucleic acid at least functionally equivalent to a nucleic acid identifiable by a signature sequence as shown in Table 1, 2 or 3.

17. The process of claim 16 wherein said antagonist is an antibody or functional fragment or functional equivalent thereof.

18. An antagonist4rected against a proteinaceous substance derived from a nucleic acid at least functionally equivalent to a nucleic acid identifiable by a signature sequence as shown in Table 1, Table 2 or Table 3.

19. The antagonist of claim 18 comprising an antibody or functional equivalent or functional fragment thereof.

20. A medicament comprising the antagonist of claim 19.

21. A method for treating an undesired immune response observed with airway hyperresponsiveness and/or bronchoalveolar manifestations of asthma in a subject, said method comprising administering the antagonist of claim 18 or claim 19 to the subject in a therapeutically effective amount and in a pharmaceutically effective manner.

22. A method for at least in part decreasing at least one symptom in a mammal suffering from an allergy or asthma, said method comprising:
blocking OtS1-B7 or an equivalent of OtS1-B7 in the mammal.

23. The method according to claim 22, wherein the OtS1-B7 is blocked by administration of a a proteinaceous substance to the mammal.

24. The method according to claim 23, wherein the proteinaceous substance is selected from the group consisting of an antibody, a functional equivalent thereof, a functional fragment thereof, and mixtures thereof.

25. The method according to claim 24, wherein the proteinaceous substance is antibody ERTR9.

26. The method according to claim 22, claim 23, claim 24, or claim 25, wherein the at least one symptom is airway hyperreactivity associated with asthma or an elevated level of IgE in the mammal.

27. The method according to claim 22, claim 23, claim 24, claim 25 or claim 26, wherein said mammal is a human.

28. A pharmaceutical composition comprising:
a substance capable of blocking OtS1-B7 or an equivalent of OtS1-B7, and a pharmaceutical acceptable carrier and/or diluent.

29. The pharmaceutical composition of claim 28, wherein the substance is a proteinaceous substance.

30. The pharmaceutical composition of claim 29, wherein the proteinaceous substance is an antibody or functional fragment thereof.

31. The pharmaceutical composition of claim 30, wherein the proteinaceous substance is antibody ERTR9.
